(19) United States
(12) Patent Application Publication
(10) Pub. No.: US 2024/0241110 A1
(43) Pub. Date: Jul. 18, 2024

(54) CHARACTERIZATION OF GLYCANS AND GLYCOCONJUGATES

(71) Applicant: Nautilus Subsidiary, Inc., Seattle, WA (US)

(72) Inventor: Parag MALLICK, San Mateo, CA (US)

(21) Appl. No.: 18/533,979

(22) Filed: Dec. 8, 2023

Related U.S. Application Data
(60) Provisional application No. 63/479,704, filed on Jan. 12, 2023.

(51) Int. Cl. G01N 33/53; G16C 20/30; G16C 20/70

(52) U.S. Cl. CPC G01N 33/5308; G16C 20/30; G16C 20/70

(57) ABSTRACT

A method, including (a) providing an array of extant glycans or glycoconjugates, wherein the array comprises a plurality of addresses, wherein different extant glycans or glycoconjugates are attached to different addresses of the array; (b) contacting the array with a plurality of different probes, the different probes recognizing different carbohydrate moieties; (c) detecting positive recognition outcomes of the plurality of different probes at individual addresses of the array, thereby producing outcome profiles for the addresses; (d) providing a database comprising a set of candidate glycans or glycoconjugates, the database comprising, for each candidate glycan or glycoconjugate, the probability of a positive recognition outcome for the plurality of different probes; and (e) determining with a computer, using the database and the outcome profiles, candidate glycans or glycoconjugates in the database corresponding to different extant glycans or glycoconjugates in the array.

### CROSS-REFERENCE TO RELATED APPLICATIONS

This application claims priority to U.S. Provisional Application No. 63/479,704, filed on Jan. 12, 2023, which is incorporated herein by reference in its entirety.

### BACKGROUND

As vital constituents of all living systems, glycans are involved in recognition, adherence, motility, and signaling processes. For example, glycoproteins play important roles in the functioning of the immune system whether biological or pathological in nature. Glycans function as binding ligands for innate immune receptors, such as selectins, galectins, and Siglecs, many of which are targets for cancer immunotherapy. Glycans impact the conformation of proteins to which they are attached and also impact protein-protein interactions. Immune checkpoint molecules PD-1 and PD-L1 are both heavily glycosylated, and the glycans facilitate their normal interactions and subsequent suppression of T cell activities. Moreover, glycans in humans are targets for the surface receptors of pathogens, such as bacteria, fungi and viruses that are involved in many infectious disease processes.

Identifying the structural differences between glycans can provide key insight into their functions. However, glycans are very diverse in nature, with over 160,000 glycan structures listed in the GlyTouCan Glycan repository. Moreover, it has been estimated that 50% or more of all proteins are glycoproteins which carry attached glycan moieties. The structures for most glycoproteins are unknown and currently available tools for structure determination are too highly specialized, costly and time consuming to provide for high throughput analysis. What is needed are methods that provide for analysis that is amenable to broader research applications, and perhaps eventual clinical application. The present disclosure satisfies this need and provides other advantages as well.

### SUMMARY

The present disclosure provides a method of characterizing glycans. The method can include steps of (a) providing an array of extant glycans, wherein the array includes a plurality of addresses, wherein different extant glycans are attached to different addresses of the array; (b) contacting the array with a plurality of different probes, the different probes recognizing different carbohydrate moieties; (c) detecting positive recognition outcomes of the plurality of different probes at individual addresses of the array, thereby producing outcome profiles for the addresses; (d) providing a database including a set of candidate glycans, the database including, for each candidate glycan, the probability of a positive recognition outcome for the plurality of different probes; and (c) determining with a computer, using the database and the outcome profiles, candidate glycans in the database corresponding to different extant glycans in the array.

Also provided is a method of characterizing glycoconjugates. The method can include steps of (a) providing an array of extant glycoconjugates, wherein the array includes a plurality of addresses, wherein different extant glycoconjugate are attached to different addresses of the array; (b) contacting the array with a plurality of different probes; (c) detecting positive recognition outcomes for the plurality of different probes at individual addresses of the array, thereby producing outcome profiles for the addresses; (d) providing a database including a set of candidate glycans or glycoconjugates, the database including, for each candidate glycan or glycoconjugate, the probability of a positive recognition outcome for the plurality of different probes; and (c) determining with a computer, using the database and the outcome profiles, candidate glycans or glycoconjugates in the database corresponding to glycoconjugates at addresses of the array.

A method of characterizing glycans can include steps of (a) providing an array of extant glycans, wherein the array includes a plurality of addresses, wherein different extant glycans are attached to different addresses of the array; (b) contacting the array with a plurality of different probes, the different probes recognizing different carbohydrate moieties; (c) detecting positive recognition outcomes of the plurality of different probes at individual addresses of the array, thereby producing outcome profiles for the addresses; (d) contacting the array with a glycan modifying reagent; (e) contacting the array with a second plurality of different probes, the different probes recognizing different carbohydrate moieties; (f) detecting positive recognition outcomes of the second plurality of different probes at individual addresses of the array, thereby producing second outcome profiles for the addresses; (g) providing a database including a set of candidate glycans, the database including, for each candidate glycan, the probability of a positive recognition outcome for the plurality of different probes; and (h) determining with a computer, using the database and the outcome profiles, candidate glycans in the database corresponding to different extant glycans in the array.

A method of characterizing glycoconjugates can include steps of (a) providing an array of extant glycoconjugates, wherein the array includes a plurality of addresses, wherein different extant glycoconjugates are attached to different addresses of the array; (b) contacting the array with a plurality of different probes, the different probes recognizing different moieties of the glycoconjugate; (c) detecting positive recognition outcomes of the plurality of different probes at individual addresses of the array, thereby producing outcome profiles for the addresses; (d) contacting the array with a glycoconjugate modifying reagent; (e) contacting the array with a second plurality of different probes, the different probes recognizing different glycoconjugate moieties; (f) detecting positive recognition outcomes of the second plurality of different probes at individual addresses of the array, thereby producing second outcome profiles for the addresses; (g) providing a database including a set of candidate glycoconjugates, the database including, for each candidate glycoconjugate, the probability of a positive recognition outcome for the plurality of different probes; and (h) determining with a computer, using the database and the outcome profiles, candidate glycoconjugates in the database corresponding to different extant glycoconjugates in the array.

The present disclosure provides a system for detecting glycans. The system can include (a) a detector configured to acquire signals from a plurality of interactions occurring between a plurality of different probes and a plurality of different extant glycans in a sample; (b) a database including information characterizing or identifying a plurality of candidate glycans; (c) a computer processor configured to: (i) communicate with the database, (ii) process the signals to produce a plurality of outcome profiles, wherein each of the outcome profiles includes a plurality of recognition outcomes for interaction of an extant glycan of (a) to the plurality of different probes, wherein individual recognition outcomes of the plurality of recognition outcomes include a measure of interaction between an extant glycan of (a) and a different probe of the plurality of different probes, (iii) process the recognition profiles to determine a probability for each of the probes interacting with each of the candidate glycans in the database according to an interaction model for each of the probes; and (iv) output an identification of selected candidate glycans, the selected candidate glycans being candidate glycans in the database having a probability for interaction with each of the probes that is most compatible with the plurality of recognition outcomes for the extant glycans.

The present disclosure further provides a system for detecting glycoproteins. The system can include (a) a detector configured to acquire signals from a plurality of binding reactions occurring between a plurality of different carbohydrate binding reagents and a plurality of different extant glycoproteins in a sample; (b) a database including information characterizing or identifying a plurality of candidate glycans; (c) a computer processor configured to: (i) communicate with the database, (ii) process the signals to produce a plurality of outcome profiles, wherein each of the binding outcome profiles includes a plurality of binding outcomes for binding of an extant glycoprotein of (a) to the plurality of different carbohydrate binding reagents, wherein individual binding outcomes of the plurality of binding outcomes include a measure of binding between an extant glycoprotein of (a) and a different carbohydrate binding reagents of the plurality of different carbohydrate binding reagents, (iii) process the binding profiles to determine a probability for each of the carbohydrate binding reagents binding to each of the candidate glycans in the database according to a binding model for each of the carbohydrate binding reagents; and (iv) output an identification of selected candidate glycans, the selected candidate glycans being candidate glycans in the database having a probability for interaction with each of the carbohydrate binding reagents that is most compatible with the plurality of binding outcomes for the extant glycoproteins.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1 shows an exemplary computer system 1001.

### DETAILED DESCRIPTION

A glycan or glycoconjugate (e.g. glycoprotein, proteoglycan or glycolipid) can be detected using one or more probes having known or measurable propensity for interaction with the glycan or glycoconjugate. For example, a carbohydrate binding reagent can bind a glycan to form a complex and a signal produced by the complex can be detected. A glycan that is detected by binding to a known carbohydrate binding reagent can be identified based on the known or predicted binding characteristics of the reagent. For example, a carbohydrate binding reagent that is known to selectively bind a candidate glycan or glycoconjugate suspected of being in a sample, without substantially binding to other glycans or glycoconjugates in the sample, can be used to identify the candidate glycan or glycoconjugate merely by observing the binding event. This one-to-one correlation of reagent binding to candidate glycan or glycoconjugate can be used for identification of one or more glycans or glycoconjugates. However, as the glycan or glycoconjugate complexity (i.e. the number and variety of different glycans or glycoconjugates) in a sample increases, the time and resources to produce a commensurate variety of probes having one-to-one specificity for the glycans or glycoconjugates approaches limits of practicality.

The present disclosure provides methods, systems and compositions that can be advantageously employed to overcome these constraints. In particular configurations, the number of different glycans or glycoconjugates identified can exceed the number of probes used. For example, the number of glycans or glycoconjugates identified can be at least 5×, 10×, 25×, 50×, 100× or more than the number of probes used. As set forth in further detail herein, one or more extant glycans or glycoconjugates can be identified by (1) performing binding reactions using promiscuous probes that interact with multiple different candidate glycans or glycoconjugates suspected of being present in a given sample, (2) subjecting one or more extant glycans or glycoconjugates to a set of the promiscuous probes that, taken as a whole, produce an empirical outcome profile for each extant glycan or glycoconjugate, and (3) performing a decoding method that evaluates the empirical outcome profile according to a trained model for interaction of the promiscuous probes with a plurality of candidate glycans or glycoconjugates, thereby identifying individual extant glycans or glycoconjugates based on compatibility of results from the model with results expected for a respective candidate glycan or glycoconjugate.

Promiscuity of a probe is a characteristic that can be understood relative to a given population of glycans. Promiscuity can arise due to a carbohydrate binding reagent recognizing an epitope that is known to be present in a plurality of different candidate glycans or glycoconjugates. For example, epitopes for which relatively few saccharide units are sufficient for recognition, such as disaccharides, trisaccharides, or tetrasaccharides, are expected to occur in a substantial number of different glycans or glycoconjugates in a typical biological system. Alternatively or additionally, a promiscuous carbohydrate binding reagent can recognize different epitopes (i.e. epitopes having a variety of different structures), the different epitopes being present in a plurality of different candidate glycans or glycoconjugates. For example, a promiscuous carbohydrate binding reagent that is designed or selected for its affinity toward a first disaccharide epitope may bind to a second epitope that has a different linkage between the monosaccharide moieties of the epitope when compared to the linkage in the first epitope. The linkages can differ with regard to chemical structure (e.g. different atoms or bonds present in the linkage), chemical reactivity, bond chirality (e.g. different enantiomers or isomers), or the like.

Although performing a single reaction between a promiscuous probe and a plurality of glycans or glycoconjugates may yield ambiguous results regarding the identity of the different glycans or glycoconjugates to which it binds, the ambiguity can be resolved when the results are combined with other identifying information. The other identifying information can include results of interactions with other promiscuous probes. For example, a plurality of different promiscuous probes can be contacted with glycans or glycoconjugates derived from a complex sample, wherein the plurality of probes is configured to produce a different outcome profile for each candidate glycan or glycoconjugate suspected of being present in the sample. In this example, each of the probes is distinguishable from the other probes, for example, in the case of probes that are carbohydrate binding reagents, due to unique labeling (e.g. different reagents have different luminophore labels), unique spatial location (e.g. different reagents are located at different sites in an array), and/or unique time of use (e.g. different reagents are delivered in series to a population of glycans or glycoconjugates). Accordingly, a plurality of promiscuous carbohydrate binding reagents produces an outcome profile for each extant glycan or glycoconjugate that can be decoded to identify a unique combination of epitopes present in the extant glycan or glycoconjugate, and this can in turn be used to identify the extant glycan or glycoconjugate as a particular candidate glycan or glycoconjugate having the same or similar unique combination of epitopes. The outcome profile can include observed binding events as well as observed non-binding events and this information can be compared to the presence and absence of epitopes, respectively, in a given candidate glycan or glycoconjugate to make a positive identification.

A plurality of recognition outcomes obtained from measuring interaction of a plurality of probes with one or more extant glycan or glycoconjugate can be input into a decoding method to identify the most likely identity of that glycan or glycoconjugate among a set of candidate glycans or glycoconjugates. The plurality of recognition outcomes can be input into a decoding method along with information characterizing or identifying the plurality of candidate glycans or glycoconjugates, and a trained model. The probability of each probe interacting with every candidate glycan or glycoconjugate in the set can be evaluated using the trained model and the decoding method can output the identity of individual extant glycans or glycoconjugates. For example, the decoding algorithm can output the most likely identity for an individual extant glycan or glycoconjugate as the candidate glycan or glycoconjugate that is most compatible with the observed recognition outcomes for the extant glycan or glycoconjugate according to the trained model.

A trained model of the present disclosure can be configured on an assumption that the characteristics for probes interacting with extant glycans or glycoconjugates in a sample, even if unknown, can be treated as quantifiable random variables, and that uncertainty about the interactions can be described by probability distributions. Parameters for a plurality of probes can be determined, for example, based on a priori knowledge about the probes (e.g. expected binding affinity of carbohydrate binding reagents for particular epitopes) and/or based on preliminary reactions performed between probes and glycans (e.g. measurement of binding between carbohydrate binding reagents and one or more epitopes). The parameters of the probes can be treated as ‘priors’ that are input into a decoding algorithm of the present disclosure. The parameters of the probes when combined with empirically determined recognition outcomes and evaluated using a decoding method of the present disclosure can output a ‘posterior,’ the calculation of which involves computation of a distribution of likelihoods for the identity of each extant glycan or glycoconjugate used for the empirical determination. The posteriors that are output by the decoding method can be used to update the priors that will be used as inputs to subsequent evaluations using the decoding method. Accordingly, the influence of unknowns and artifacts in early evaluation of affinity reagents can be diminished as further empirical measurements are made and the results evaluated by the decoding method. This updating cycle can provide the benefit of facilitating iterative improvement to the decoding method, thereby improving the accuracy of identifying or characterizing extant glycans or glycoconjugates.

An advantage of the decoding approach set forth herein is that it takes into account characteristics of single-analyte reactions that may otherwise adversely affect the accuracy with which glycans or glycoconjugates can be identified. For example, binding reactions carried out at single-molecule scale (e.g. detecting binding of a carbohydrate binding reagent to a glycoconjugate that is individually resolved on a glycoconjugate array) produce stochastic results. A decoding method can be configured to account for stochasticity, non-specific binding, or other factors for improved accuracy when identifying or characterizing glycans or glycoconjugates. For example, a decoding algorithm or trained model used by the algorithm can apply a non-zero probability of a given probe reacting with each candidate glycan or glycoprotein suspected of being in an assay. The same non-zero probability can be applied to all candidate glycans or glycoproteins in a set or different candidates can have a different non-zero probabilities, for example, based on a priori characteristics of the respective candidates or based on empirical measurements for the candidates.

The present disclosure provides compositions, systems and methods that can be useful in various configurations for characterizing glycans or glycoconjugates by obtaining multiple separate and non-identical measurements of their interactions with a plurality of different probes. In particular configurations, the individual measurements may not, by themselves, be sufficiently accurate or specific to make the characterization, but an aggregation of the multiple non-identical measurements can allow the characterization to be made with a high degree of accuracy, specificity and/or confidence. Optionally, a plurality of promiscuous probes can be reacted with a given glycan or glycoconjugate and the recognition outcome observed for each of the promiscuous probes can be detected. Promiscuous probes can demonstrate both low specificity, with regard to the variety of different glycans or glycoconjugates recognized, and high reactivity for some or all of those analytes. Taking a binding reaction as an example, promiscuous carbohydrate binding reagents can demonstrate both low specificity, with regard to the variety of different glycans recognized, and high affinity for some or all of the glycans that are recognized. For any of a variety of reactions, including but not limited to binding reactions, a first reaction carried out using a first promiscuous probe may perceive a first subset of glycans or glycoconjugates in a sample without distinguishing one glycan or glycoconjugate in the subset from other glycans or glycoconjugates in the sample. A second reaction carried out using a second promiscuous probe may perceive a second subset of glycans or glycoconjugates in the sample, again, without distinguishing one glycan or glycoconjugate from other glycans or glycoconjugates in the sample. However, a combination of measurements obtained from the first and second reactions can distinguish: (i) a glycan or glycoconjugate that is present in the first subset but not the second; (ii) a glycan or glycoconjugate that is present in the second subset but not the first; (iii) a glycan or glycoconjugate that is present in both the first and second subsets; or (iv) a glycan or glycoconjugate that is absent in the first and second subsets. The number of promiscuous probes used, the number of separate measurements acquired, and degree of probe promiscuity (e.g. the diversity of components recognized by the probe) can be adjusted to suit the known or suspected diversity of different glycans or glycoconjugates for a given sample.

A composition, system or method set forth herein can be used to characterize a glycan or glycoconjugate with respect to any of a variety of characteristics or features including, for example, presence, absence, quantity (e.g. amount or concentration), chemical reactivity, molecular structure, structural integrity (e.g. full-length or fragmented), location (e.g. in an analytical system such as an array, subcellular compartment, cell or natural environment), association with another analyte or moiety, binding affinity for another analyte or moiety, biological activity, chemical activity or the like. A glycan or glycoconjugate can be characterized with regard to a relatively generic characteristic such as the presence or absence of a common structural feature (e.g. an oligosaccharide subunit, a particular linkage between saccharide subunits, or an enantiomer). A characterization can be sufficiently specific to identify a glycan or glycoconjugate, for example, at a level that is considered adequate or unambiguous by those skilled in the art. A glycan or glycoconjugate can be identified with a probability or score surpassing a desired threshold for confident identification.

Methods, compositions and systems of the present disclosure can be advantageously deployed in situations where glycans yield different empirical binding profiles despite having identical compositions and being subjected to the same set of probes. For example, the methods, compositions and systems are well suited for single-molecule detection and other formats that are prone to stochastic variability. Particular configurations of the compositions, systems and methods herein can overcome ambiguities and errors in observed recognition outcomes to provide accurate identification and characterizations of glycans or glycoconjugates. The methods can be advantageously deployed for complex samples including glycoproteomes, cell surfaces or subfractions thereof.

Terms used herein will be understood to take on their ordinary meaning in the relevant art unless specified otherwise. Several terms used herein and their meanings are set forth below.

As used herein, the term “address” refers to a location in an array where a particular analyte (e.g. glycan, glycoprotein, or cell) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “array” refers to a population of analytes (e.g. glycoconjugates or glycans) that are associated with unique identifiers such that the analytes can be distinguished from each other. A unique identifier can be, for example, a solid support (e.g. particle or bead), spatial address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “carbohydrate binding reagent” refers to a molecule or other substance that is capable of specifically or reproducibly forming a complex with a carbohydrate molecule or carbohydrate moiety. A carbohydrate binding reagent may form a reversible or irreversible bond with a carbohydrate. A carbohydrate binding reagent may bind with a carbohydrate in a covalent or non-covalent manner. Carbohydrate binding reagents may include reactive reagents, catalytic reagents, or non-reactive reagents. A carbohydrate binding reagent can be non-reactive and non-catalytic, thereby not permanently altering the chemical structure of a carbohydrate to which it binds. Carbohydrate binding reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins, sulfated glycosaminoglycan-binding proteins or functional fragments thereof.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “fluid-phase,” when used in reference to a molecule, means the molecule is in a state wherein it is mobile in a fluid, for example, being capable of diffusing through the fluid.

As used herein, the term “glycan” refers to a molecule or moiety consisting of a multiple monosaccharides linked glycosidically. Exemplary glycans include saccharide polymers such as disaccharides, oligosaccharides, or polysaccharides. A glycan can be a moiety of a glycoconjugate such as a glycolipid, glycoprotein or proteoglycan.

As used herein, the term “glycoprotein” refers to a molecule containing a carbohydrate covalently linked to protein. The carbohydrate may be in the form of a glycan, monosaccharide, or saccharide polymer such as an oligosaccharide or polysaccharide. One or more carbohydrate units may be present. An exemplary glycan is an N-linked glycan moiety which can be attached to the nitrogen in the side chain of an asparagine (Asn) of a protein moiety. The N-linked glycan asparagine may be present in a sequon (i.e. Asn-X-Ser or Asn-X-Thr sequences, where X is any amino acid except proline). An N-linked glycan may optionally be composed of N-acetylgalactosamine, galactose, neuraminic acid, N-acetylglucosamine, fucose, mannose, or other monosaccharides. Another exemplary saccharide polymer that can be present in a glycoprotein is an O-linked glycan which can be attached to the oxygen in the side chain of a serine or threonine residue. Proteoglycans are a type of glycoprotein in which the carbohydrate units are polysaccharides that contain amino sugars.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “origami,” when used in reference to a nucleic acid, refers to a construct of the nucleic acid having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “outcome profile” refers to a plurality of recognition outcomes for a glycan or other analyte. The recognition outcomes in the set can be obtained from independent observations, for example, independent binding outcomes can be acquired using different carbohydrate binding reagents, respectively, or using different affinity reagents, respectively. Alternatively, the recognition outcomes can be statistical measures such as probabilities, likelihoods, measures of uncertainty or measures of variation. Optionally, the recognition outcomes can be generated in silico, for example, being derived from a modification of an empirically obtained recognition outcome. An outcome profile can include empirical outcomes, candidate outcomes, calculated outcomes, theoretical outcomes or a combination thereof. An outcome profile can include a vector of recognition outcomes. The elements of the vector can be digital values (e.g. binary values representing positive and negative binding outcomes respectively) or continuous values (e.g. probability values in a range from 0 to 1). The term ‘continuous values’ can refer to a range having an unspecified number of values, for example, non-binary values or non-digital values. The terms ‘continuous’ and ‘analog,’ when used in reference to values or numbers, are intended to be synonymous herein and in U.S. Pat. App. Ser. No. 63/479,704.

As used herein, the term “promiscuous,” when used in reference to a reagent, means that the reagent is known or suspected to react with a variety of different analytes in a given sample. For example, a carbohydrate binding reagent that is known or suspected to recognize a variety of different carbohydrate moieties (e.g. a variety of different disaccharides or a variety of different trisaccharides) is promiscuous. A promiscuous reagent may be known or suspected of having high reactivity with one or more of the different analytes with which it reacts. For example, a promiscuous carbohydrate binding reagent may have high affinity for one or more of the different analytes that it recognizes. A promiscuous reagent may be composed of a single species of reagent, such as a single carbohydrate binding reagent, or a promiscuous reagent may be composed of two or more different reagent species. For example, a promiscuous carbohydrate binding reagent may be composed of a single species of lectin that recognizes a variety of different carbohydrate moieties in a sample, or a promiscuous carbohydrate binding reagent may be composed of a pool containing several different lectin species that collectively recognize a variety of different carbohydrate moieties in a sample.

As used herein, the term “protein” refers to a molecule or moiety comprising two or more amino acids joined by a peptide bond. A protein may also be referred to as a polypeptide, oligopeptide or peptide. A protein can be naturally occurring or synthetic. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “recognition outcome” refers to information resulting from observation, simulation or examination of an analytical process. For example, the recognition outcome for contacting a carbohydrate binding reagent with a glycan can be referred to as a “binding outcome.” Similarly, the recognition outcome for contacting an affinity reagent with a protein can also be referred to as a “binding outcome.” A recognition outcome can be positive or negative. For example, observation of binding is a positive binding outcome and observation of non-binding is a negative binding outcome. A recognition outcome can be a null outcome in the event a positive or negative outcome is not apparent. A recognition outcome is “empirical” when it includes information based on observation of a signal from an analytical technique. A “candidate” recognition outcome can include an empirical or putative recognition outcome for a candidate analyte (e.g. for a candidate glycan or candidate protein) that is known or suspected of being present in a sample or assay. A recognition outcome can be represented in binary terms such as a zero (0) for a negative binding outcome and a one (1) for a positive binding outcome. In some cases, a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different recognition outcomes.

As used herein, the term “single,” when used in reference to an object such as an analyte, means that the object is individually manipulated or distinguished from other objects. A single analyte can be a single molecule (e.g. single glycan), a single complex of two or more molecules (e.g. a multimeric protein having two or more separable subunits, a single protein attached to a structured nucleic acid particle or a single glycan attached to a lectin), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as polypeptides, reactive moieties, or detectable labels) or are configured to be attached to other molecules (e.g., by functional groups). Exemplary SNAPs include nucleic acid origami and nucleic acid nanoballs.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

The embodiments set forth below and recited in the claims can be understood in view of the above definitions.

The present disclosure provides a method of characterizing glycans. The method can include steps of (a) providing an array of extant glycans, wherein the array includes a plurality of addresses, wherein different extant glycans are attached to different addresses of the array; (b) contacting the array with a plurality of different probes, the different probes recognizing different carbohydrate moieties; (c) detecting positive recognition outcomes of the plurality of different probes at individual addresses of the array, thereby producing outcome profiles for the addresses; (d) providing a database including a set of candidate glycans, the database including, for each candidate glycan, the probability of a positive recognition outcome for the plurality of different probes; and (c) determining with a computer, using the database and the outcome profiles, candidate glycans in the database corresponding to different extant glycans in the array.

Also provided is a method of characterizing glycoconjugates. The method can include steps of (a) providing an array of extant glycoconjugates, wherein the array includes a plurality of addresses, wherein different extant glycoconjugate are attached to different addresses of the array; (b) contacting the array with a plurality of different probes; (c) detecting positive recognition outcomes for the plurality of different probes at individual addresses of the array, thereby producing outcome profiles for the addresses; (d) providing a database including a set of candidate glycans or glycoconjugates, the database including, for each candidate glycan or glycoconjugate, the probability of a positive recognition outcome for the plurality of different probes; and (c) determining with a computer, using the database and the outcome profiles, candidate glycans or glycoconjugates in the database corresponding to glycoconjugates at addresses of the array.

A glycan that is made or used as set forth herein can be a carbohydrate molecule or a carbohydrate moiety of a glycoconjugate. A glycan molecule can lack non-carbohydrate moieties. A glycan molecule can be in the form of a free reducing glycan. Glycoconjugates include a glycan moiety attached to a non-carbohydrate moiety. For example, a glycoprotein or proteoglycan can include at least one glycan moiety covalently attached to at least one protein moiety. A glycolipid can include at least one glycan moiety covalently attached to a lipid moiety. A glycan can also be attached to a cell, tissue or other biological entity.

A glycan or glycoconjugate can be derived from a natural or synthetic source. Exemplary sources include, but are not limited to a biological tissue, fluid, cell or subcellular compartment (e.g. organelle). For example, a sample can be derived from a tissue biopsy, biological fluid (e.g. blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or polypeptide synthesis reaction. Any sample where a glycan is a native or expected constituent can be used.

Exemplary organisms from which a glycan or glycoconjugate can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharomyces cerevisiae or Schizosaccharomyces pombe; or a Plasmodium falciparum. A polypeptide can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A glycan can be derived from a homogeneous culture or population of the above organisms, or alternatively from a collection of several different organisms, for example, in a community or ecosystem.

In some cases, a glycan or glycoconjugate can be derived from an organism that is collected from a host organism. A glycan may be derived from a parasitic, pathogenic, symbiotic, or latent organism collected from a host organism. A glycan can be derived from an organism, tissue, cell or biological fluid that is known or suspected of being associated with a disease state or disorder (e.g., an oncogenic virus). Alternatively, a glycan can be derived from an organism, tissue, cell or biological fluid that is known or suspected of not being associated with a particular disease state or disorder. For example, one or more glycan isolated from such a source can be used as a control for comparison to results acquired from a source that is known or suspected of being associated with the particular disease state or disorder. A sample may include a microbiome. A sample may include a plurality of glycans contributed by microbiome constituents. In some cases, one or more glycans used in a method, composition or apparatus set forth herein may be obtained from a single organism (e.g. an individual human), single cell, single organelle, or single polypeptide-containing particle (e.g., a viral particle).

A glycan or glycoconjugate can optionally be separated or isolated from other components of the source from which it is derived. For example, a glycan or glycoconjugate can be separated or isolated from lipids, nucleic acids, hormones, enzyme cofactors, vitamins, metabolites, microtubules, organelles (e.g. nucleus, mitochondria, chloroplast, endoplasmic reticulum, vesicle, cytoskeleton, vacuole, lysosome, cell membrane, cytosol or Golgi apparatus), polypeptides or the like. A glycoconjugate can be separated or isolated from one or more non-glycoconjugates. For example, a glycoprotein, such as a proteoglycan, can be separated or isolated from proteins that are not conjugated to glycan moieties. Separation of proteins such as glycoproteins can be carried out using methods known in the art such as centrifugation (e.g. to separate membrane fractions from soluble fractions), density gradient centrifugation (e.g. to separate different types of organelles), precipitation, affinity capture, adsorption, liquid-liquid extraction, solid-phase extraction, chromatography (e.g. affinity chromatography, ion exchange chromatography, reverse phase chromatography, size exclusion chromatography, electrophoresis (e.g. polyacrylamide gel electrophoresis) or the like. Particularly useful polypeptide separation methods are set forth in Scopes, Polypeptide Purification Principles and Practice, Springer; 3rd edition (1993). Similar techniques can be used to separate or isolate glycans or other glycoconjugates such as glycolipids. Exemplary methods for separating or isolating glycan and glycoconjugates are set forth in Zhang et al. Frontiers in Chemistry, vol 8, Article 508 (2020) doi: 10.3389/fchem.2020.00508, which is incorporated herein by reference.

Optionally, one or more glycan moieties can be released from a glycoconjugate before, during or after a one or more steps of a method set forth herein. In some configurations of methods set forth herein, glycan moieties can be released from glycoconjugates prior to attaching the glycans to a solid support or unique identifier, prior to reacting the glycans with a probe such as a carbohydrate binding reagent, and/or prior to detecting the glycans. In some configurations, glycan moieties can be released from glycoconjugates after the glycoconjugates have been attached to a solid support or unique identifier, after reacting the glycoconjugates with a probe such as a carbohydrate binding reagent, and/or after detecting the glycoconjugates. Optionally, glycan moieties can be released using techniques or reagents that are effective for one or more types of glycoconjugates. For example, N-glycans or O-glycans can be released from glycoproteins; glycans can be released from glycolipids such as glycosphingolipids; or glycans can be released from glycosaminoglycans. Glycans can be released by breaking covalent bonds that attach glycan moieties to non-glycan moieties of glycoconjugates. Exemplary methods for releasing glycans are set forth in Zhang et al. Frontiers in Chemistry, vol 8, Article 508 (2020) doi: 10.3389/fchem.2020.00508, which is incorporated herein by reference.

A glycoprotein can be in a native conformation or denatured conformation when used as set forth herein. For example, a glycoprotein can be in a native conformation, whereby it is capable of performing native function(s) such as catalysis of its natural substrates or binding to its natural substrates. Alternatively, a glycoprotein can be in a denatured conformation such that it is incapable of performing native function(s) such as catalysis of its natural substrates or binding to its natural substrates. A glycoprotein can be in a native conformation for some manipulations set forth herein and in a denatured conformation for other manipulations set forth herein. A glycoprotein may be denatured at any stage during manipulation, including for example, upon removal from a native milieu or at a later stage of processing such as a stage where the glycoprotein is separated from other cellular components, fractionated from other polypeptides, functionalized to include a reactive moiety, attached to a particle or solid support, contacted with a probe, detected, digested to produce fragments, or other step set forth herein. A denatured glycoprotein may be refolded, for example, reverting to a native state for one or more step of a process set forth herein.

A cell can include one or more glycans on its surface. In some configurations of the methods set forth herein, one or more cells can be isolated and surface glycans can be detected on the intact cells. For example, a cell can be attached to a solid support, such as an address in an array, and glycans on the cell can be detected. A plurality of cells can be separated by attachment to addresses of an array and glycans can be detected on the array. In some configurations, glycans are removed from isolated cells prior to detection. For example, cells that are known or suspected of having one or more glycan of interest can be separated from other biological components (e.g. other cells), and the glycans can then be removed from the cells. The glycans can be obtained as a membrane fraction from one or more cell, as glycoproteins fractionated from one or more cell, or as glycolipids fractionated from one or more cell. Fractions obtained from one or more cell can be attached to a solid support, such as one or more addresses of an array.

A plurality of glycans or glycoconjugates can be characterized in terms of total number of glycan or glycoconjugate molecules that are present in an assay or in a sample that is assayed. A plurality of glycan or glycoconjugate molecules used or included in a method, composition or system set forth herein can include at least 2, 10, 100, 1×10⁶, 1×10⁹ or more molecules. Alternatively or additionally, a plurality of glycan or glycoconjugate molecules may contain at most 1×10⁹, 1×10⁶, 100, 10 or 2 molecules.

A plurality of glycans or glycoconjugates can be characterized in terms of the variety of different chemical structures in the plurality. For example, plurality of glycans or glycoconjugates can have a complexity of at least 2, 5, 10, 100, 1×10³, 1×10⁶ or more different glycan or glycoconjugate structures. Alternatively or additionally, a plurality of glycans or glycoconjugates can have a complexity that is at most 1×10⁶, 1×10³, 100, 10, 5, 2 or fewer different glycan or glycoconjugate structures.

A plurality of glycans or glycoconjugates can be characterized in terms of the dynamic range for the different glycan or glycoconjugate structures in the sample. The dynamic range can be a measure of the range of abundance for all different glycan or glycoconjugate structures in a plurality of glycans or glycoconjugates, the range of abundance for all different glycan or glycoconjugate structures in a plurality of glycans or glycoconjugates, the range of abundance for all different glycan or glycoconjugate structures in a plurality of glycans or glycoconjugates, or the range of abundance for all different full-length gene products in a plurality of glycoproteins. The dynamic range for plurality of glycans or glycoconjugates can be a factor of at least 10, 100, 1×10³, 1×10⁶, 1×10⁹, or more. Alternatively or additionally, the dynamic range for plurality of glycans or glycoconjugates can be a factor of at most 1×10⁹, 1×10⁶, 1×10³, 100, 10 or less.

Detection of multiple different glycans or glycoconjugates can be performed in a multiplex format. In multiplexed formats, different glycans or glycoconjugates can be attached to different unique identifiers (e.g. sites in an array), and then manipulated or detected in parallel. For example, a fluid containing one or more different probes can be delivered to an array of addresses, each address having an immobilized glycan or glycoconjugate, such that the addresses of the array are in simultaneous contact with the probe(s). The glycans or glycoconjugates can be attached to the respective addresses via particles or, alternatively, they can be directly attached to the respective addresses. Moreover, a plurality of addresses can be observed in parallel allowing for rapid detection of glycans or glycoconjugates.

An array can include at least 10, 100, 1000, 1×10⁶, 1×10⁹ or more addresses. Alternatively or additionally, an array can include at most 1×10⁹, 1×10⁶, 1000, 100, 10 or fewer addresses. The addresses of an array can be occupied by different glycans or glycoconjugates. Some addresses can be occupied by glycans or glycoconjugates having the same structure, for example, when multiple copies of a glycan or glycoconjugate are present in a sample of interest. Accordingly, an array can include at least 10, 100, 1000, 1×10⁶, 1×10⁹ or more different glycans or glycoconjugates. Alternatively or additionally, an array can include at most 1×10⁹, 1×10⁶, 1000, 100, 10 or fewer different glycans or glycoconjugates. It will be understood that in some configurations an array can include only one type of glycan or glycoconjugate.

Glycans can be attached to a unique identifier or solid support using covalent or non-covalent bonding. Exemplary covalent attachments include chemical linkers such as those produced using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier (or solid support) and the ligand is attached to the glycan (or glycoconjugate). Alternatively, the ligand can be attached to the unique identifier (or solid support) and the receptor can be attached to the glycan (or glycoconjugate). Glycans can optionally be attached via bonding to the carbohydrate structure of the glycan. Exemplary chemistries include those used to prepare glycan arrays such as those set forth in Blixt et al., “Printed covalent glycan array for ligand profiling of diverse glycan binding proteins,” Proc. Nat'l Acad. Sci. USA 101:17033017038 (2004); and Gao et al. “Glycan microarrays as chemical tools for identifying glycan recognition by immune proteins,” Frontiers in Chemistry Vol. 7 Article 833 (2019), each of which is incorporated herein by reference. Glycoproteins can be attached to a unique identifier or solid support via a carbohydrate moiety or via a protein moiety. Exemplary reagents and methods for attaching protein moieties to an array are set forth in US Pat. App. Pub. No. 2021/0101930 A1 or U.S. patent application Ser. No. 17/692,035, each of which is incorporated herein by reference.

Optionally, a glycan or glycoconjugate can be attached to a solid support or address of an array via a particle. Structured nucleic acid particles are particularly useful such as those that include nucleic acid origami. A nucleic acid origami can include one or more nucleic acids having a variety of overall shapes such as a disk, tile, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference. In some configurations, a nucleic acid origami may include a scaffold nucleic acid and a plurality of staple nucleic acids. The scaffold can be configured as a single, continuous strand of nucleic acid, and the staples can be formed by nucleic acids that hybridize, in whole or in part, with the scaffold nucleic acid. A particle including one or more nucleic acids (e.g. as found in origami or nanoball structures) may include regions of single-stranded nucleic acid, regions of double-stranded nucleic acid, or combinations thereof.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold nucleic acid can be linear (i.e. having a 3′ end and 5′ end) or circular (i.e. closed such that the scaffold lacks a 3′ end and 5′ end). A nucleic acid scaffold can be derived from a natural source, such as a viral genome or a bacterial plasmid. For example, a nucleic acid scaffold can include a single strand of an M13 viral genome. In other configurations, a nucleic acid scaffold may be synthetic, for example, having a non-naturally occurring sequence in full or in part. A scaffold nucleic acid can be single stranded but for a plurality of oligonucleotides hybridized thereto or short regions of internal complementarity. The size of a nucleic acid scaffold may vary to accommodate different uses. For example, a nucleic acid scaffold may include at least about 100, 500, 1000, 2500, 5000 or more nucleotides. Alternatively or additionally, a nucleic acid scaffold may include at most about 5000, 2500, 1000, 500, 100 or fewer nucleotides.

A nucleic acid origami can include a plurality of oligonucleotides that are hybridized to a scaffold nucleic acid. A first region of an oligonucleotide sequence can be hybridized to a scaffold nucleic acid while a second region is not hybridized to the scaffold. One or both of the regions can be located at or near the 5′ end of the oligonucleotide, at or near the 3′ end of the oligonucleotide, or in a region of the oligonucleotide that is between the end regions. The second region can be in a single stranded state or, alternatively, can participate in a hairpin or other self-annealed structure in the oligonucleotide. Optionally, the second region of the oligonucleotide can include an attachment moiety that is configured to form a covalent or non-covalent bond with a reactive moiety, such as an amino acid of a polypeptide or a carbohydrate moiety of a glycan. The first and second regions of an oligonucleotide can be adjacent to each other in the oligonucleotide sequence or separated by a spacer region. The spacer region can be single stranded, for example, to provide relative flexibility. Alternatively, the spacer region can be double stranded or at least partially double stranded, for example, to provide relative rigidity.

An oligonucleotide can include two sequence regions that are hybridized to a scaffold nucleic acid, for example, to function as a ‘staple’ that restrains the structure of the scaffold. For example, a single oligonucleotide can hybridize to two regions of a scaffold that are separated from each other in the primary sequence of the scaffold. As such, the oligonucleotide can function to retain those two regions of the scaffold in proximity to each other or to otherwise constrain the scaffold to a desired conformation. Two sequence regions of an oligonucleotide staple can be adjacent to each other in the oligonucleotide sequence or separated by a spacer region that does not hybridize to the scaffold. One or more regions of an oligonucleotide that hybridize to a scaffold nucleic acid can be located at or near the 5′ end of the oligonucleotide, at or near the 3′ end of the oligonucleotide, or in a region of the oligonucleotide that is between the end regions. Oligonucleotides can be configured to hybridize with a nucleic acid scaffold, another oligonucleotide, a staple oligonucleotide, or a combination thereof. The oligonucleotides can be linear (i.e. having a 3′ end and a 5′ end) or closed (i.e. circular, lacking both 3′ and 5′ ends).

An oligonucleotide that is included in a nucleic acid origami can have any of a variety of lengths. An oligonucleotide may have a length of at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide in a nucleic acid origami may hybridize with another oligonucleotide or a scaffold strand forming a particular number of base pairs. An oligonucleotide may form a hybridization region of at least about 5, 6, 7, 8, 9, 10, 15, 20, 25, 50 or more consecutive or total base pairs. Alternatively or additionally, an oligonucleotide may form a hybridization region of no more than about 50, 25, 20, 15, 10, 9, 8, 7, 6, 5, or fewer consecutive or total base pairs.

A glycan or glycoconjugate can be attached to nucleic acid origami via a scaffold component or oligonucleotide component of the origami structure. For example, the scaffold or oligonucleotide can include one or more nucleotide analog(s) that attach covalently or non-covalently to a glycan or glycoconjugate. A nucleic acid origami, or other particle set forth herein, can include at least 1, 2, 5, 10, 20, 30, 40, 50, 75, 100 or more moieties of a type set forth herein (e.g. moieties that react with glycans or glycoconjugates, or label moieties). Alternatively or additionally, a nucleic acid origami, or other particle set forth herein, can include at most 100, 75, 50, 40, 30, 20, 10, 5, 2, or 1 moieties of a type set forth herein.

Another type of structured nucleic acid particle is a nucleic acid nanoball. Nucleic acid nanoballs may be fabricated by a method such as rolling circle amplification using a circular template to generate a nucleic acid amplicon consisting of a concatemer of template complements. The amplicon can be further modified to include crosslinks, for example, in the form of staples that hybridize to different regions of the amplicon. Exemplary methods for making nucleic acid nanoballs are described, for example, in U.S. Pat. No. 8,445,194, which is incorporated herein by reference. Further examples of structured nucleic acid particles that include nucleic acid origami or nanoballs are set forth, for example, in U.S. Pat. No. 11,203,612; US Pat. App. Pub. No. 2022/0162684 A1 or U.S. patent application Ser. No. 17/692,035, each of which is incorporated herein by reference.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

A particle need not be composed primarily of nucleic acid and, in some cases, may be devoid of nucleic acids. For example, a particle can be composed of a solid support material, such as a silicon or silica nanoparticle, a carbon nanoparticle, a cellulose nanobead, a PEG nanobead, a polymeric nanoparticle (e.g., polyethyleneimine, dendritic polymer, dendrimer, polyacrylate particle, polystyrene-based particle, FluoSphere™, etc.), upconversion nanocrystal, or a quantum dot. A particle may include solid materials and shell-like materials (e.g., carbon nanospheres, silicon oxide nanoshells, iron oxide nanospheres, polymethylmethacrylate nanospheres, etc.).

A particle may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to length, area, or volume. The length, area or volume can be characterized in terms of a minimum, maximum, or average for a population. Optionally, a particle can have a minimum, maximum or average length of at least about 50 nm, 100 nm, 250 nm, 500 nm, 1 mm, 5 mm or more. Alternatively or additionally, a particle can have a minimum, maximum or average length of no more than about 5 mm, 1 mm, 500 nm, 250 nm, 100 nm, 51 nm, or less. Optionally, a particle can have a minimum, maximum or average volume of at least about 1 mm³, 10 mm³, 100 mm³, 1 mm³ or more. Alternatively or additionally, a particle can have a minimum, maximum or average volume of no more than about 1 mm³, 100 mm³, 10 mm³, 1 mm³ or less.

A particle that is made or used in a method set forth herein can be suspended in a fluid, immobilized on a solid support, or immobilized in another material such as a gel or solid support material. For example, a population of particles can be colloidal for some, or all steps of a method set forth herein. Alternatively, a population of particles can be immobilized in, or on a gel or solid support for some, or all steps of a method set forth herein.

A method of the present disclosure can include a step of contacting a plurality of glycan or glycoconjugates with a plurality of different probes, the different probes recognizing different carbohydrate moieties. A plurality of glycans or glycoconjugates can be detected in a fluid or on a solid support. For fluid configurations, a fluid containing a plurality of glycans or glycoconjugates can be mixed with another fluid containing one or more probes. When on solid support, one or more glycans or glycoconjugates can be attached to a particle or solid support. Probes, such as carbohydrate binding reagents that will participate in a binding event, can be present in a fluid that is in contact with the particle or solid support. Optionally, the solid support includes an array of addresses, the glycans or glycoconjugates being attached to the addresses. Typically, the identity of the glycan or glycoconjugate at any given address is not known (as such, the glycan or glycoconjugate may be referred to as being ‘unknown’). Methods set forth herein can be used to identify glycans or glycoconjugates at one or more addresses in an array. Accordingly, the methods can be used to locate extant glycans or glycoconjugates in an array.

Particularly useful probes are carbohydrate binding reagents. Exemplary carbohydrate binding reagents include lectins, sulfated glycosaminoglycan-binding proteins, microbial adhesins, viral agglutinins, nucleic acid aptamers, antibodies or functional fragments thereof. Examples of such carbohydrate binding reagents are provided in further detail below.

A variety of lectins can be usefully applied in methods set forth herein according to the monosaccharide(s) or other carbohydrate moieties that they recognize or according to the α- or β-anomers of the carbohydrates they recognize. Lectins within a particular monosaccharide specificity group may also differ in their affinities for different glycans. For example, concanavalin A (ConA) is an α-mannose/α-glucose-binding lectin that recognizes N-glycans and is not known to bind common O-glycans on animal cell glycoproteins. However, it binds oligomannose-type N-glycans with substantially higher affinity than complex-type biantennary N-glycans, and it does not recognize more highly branched complex-type N-glycans. Other lectins, such as L-phytohemagglutinin (L-PHA) and E-PHA from Phaseolus vulgaris, as well as lentil lectin (LCA) from Lens culinaris, also recognize specific determinants of N-glycans. Some animal lectins that are widely used include those from invertebrates, such as Helix pomatia agglutinin (HPA) from snail.

Recombinant lectins, including engineered varieties can be particularly useful. Structural engineering of lectins is benefited from the ever-increasing number of crystal structures of oligosaccharide-protein complexes. Recombinant lectins derived from bacterial adhesins (e.g., “Siglec-like” binding regions from Streptococcus mutans) can be engineered to have a variety of altered specificities for carbohydrate epitopes. Recombinant techniques can also be used to produce inactivated enzymes that are capable of binding to glycans. For example, engineered glycan binding reagents can be derived from inactivated glycosylhydrolases and other enzymes. Inactivation, for example, by point mutation of a catalytic amino acid can produce a probe that retains affinity for glycans to be useful as a detection probe. As an example, an inactivated bacteriophage-derived endosialidase can have lectin-like properties and can be used as a probe in the specific detection of its substrate, polysialic acid. See Varki et al., Essentials of Glycobiology 4th ed. Cold Spring Harbor (NY): Cold Spring Harbor Laboratory Press (2022) Chapter 48, which is incorporated herein by reference.

Exemplary lectins that can be used to detect glycans include, but are not limited to, concanavalin A (ConA) which can recognize oligomannose-type N-glycan, hybrid type N-glycan or biantennary complex-type N-glycan; Galanthus nivalis agglutinin (GNA) which can recognize terminal alpha1-3-linked mannose; Phaseolus vulgaris erythroagglutinin (E-PHA) which can recognize bisected bi-, triantennary complex-type N-glycan or 2,6-branched tetraantennary complex-type N-glycan; Datura stramonium agglutinin (DSA) which can recognize branched complex-type N-glycan with poly-N-acetyllactosamine; Ricinus communis agglutinin-I (RCA-I) which recognizes beta-linked terminal Gal; Erythrina cristigalli lectin (ECL) which recognizes beta1-4-linked terminal Gal; Griffonia simplicafolia agglutinin I-B4 (GS-I-B4) which recognizes alpha1-3-linked terminal Gal; Wisteria floribunda agglutinin (WFA) which recognizes terminal GalNAc; Lycopersicon esculentum agglutinin (LEA) which recognizes linear poly-N-acetyl lactosamine; Solanum tuberosum lectin which recognizes linear poly-N-acetyl lactosamine; Datura stramonium agglutinin which recognizes linear poly-N-acetyl lactosamine; Phytolacca americana mitogen which recognizes branched poly-N-acetyl lactosamine; Triticum vulgaris agglutinin which recognizes branched poly-N-acetyl lactosamine; Ulex europaeus agglutinin (UEA-I) which recognizes alpha1-2-linked fucose and H-type 2 Galbeta1-4GlcNAc; Anguilla anguilla agglutinin (AAA) which recognizes alpha1-2-linked fucose; Aleuria aurantia lectin (ALL) which recognizes alpha1-6 linked fucose; Lens culinaris agglutinin (LCA) which recognizes bi-,triantennary core alpha6-fucosylated complex N-glycans; Pisum sativum agglutinin which recognizes bi-,triantennary core alpha6-fucosylated complex N-glycans; Maackia amurensis erythroagglutinin (MAH) which recognizes alpha2-3-sialylated core 1,3-O-sulfated LacNAc, or alpha2-3-linked sialic acid; CBM40 which recognizes alpha2-3-linked sialic acid; Sambucus nigra agglutinin (SNA) which recognizes alpha2-6-linked sialic acid, 6-O-sulfated LacNac or sialylated Tn antigen; Triticum vulagaris agglutinin which recognizes sialylated glycans or terminal GlcNAc; Helix pomatia agglutinin (HPA) which recognizes terminal alpha-GlcNAc; Vicia villosa agglutinin (VVA) which recognizes terminal alpha-GlcNAc; Wisteria floribunda agglutinin (WFA) which recognizes terminal alpha-GlcNAc; Dolichus biflorus agglutinin (DBA) which recognizes terminal alpha-GlcNAc; Arachis hypogaea agglutinin which recognizes core 1 (T antigen); or Artocarpus integrifolia agglutinin which recognizes core 1 (T antigen) or core 3. See Varki et al., Essentials of Glycobiology 4th ed. Cold Spring Harbor (NY): Cold Spring Harbor Laboratory Press (2022) Chapter 48, which is incorporated herein by reference.

Antibodies and functional fragments thereof (e.g., Fab′ fragments, F(ab′)₂ fragments, single-chain variable fragments (scFv), di-scFv, tri-scFv, or microantibodies) can be raised against glycans. For example, antibodies or functional fragments thereof can be obtained using whole cells, cell-derived membrane fractions, isolated glycoproteins, isolated glycolipids, isolated glycan, synthetic glycoproteins, synthetic glycolipids, synthetic glycans or carbohydrates to immunize antibody-producing organisms (e.g. mammals such as mice or rabbits, or sea lamprey) or as targets for selection or screening (e.g. for phage display). Antibodies or functional fragments are available in the art that are capable of recognizing glycan epitopes such as Sialyl-Lewis x (SLe^{x}), Sialyl-Lewis a (SLe^{a}), Lewis x (Le^{x}), Lewis a (Le^{a}), Lewis y (Le^{y}), Lewis b (Le^{b}), 3′-sulfo-Lewis x, 3′-sulfo-Lewis a, 6-sulfo-sialyl-Lewis x, 6-sulfo-sialyl-Lewis a, MECA-79, VIM-2, sialyl T antigen, sialyl Tn antigen, T antigen, Tn antigen, type 1 H antigen, type 1 A antigen, type 1 B antigen, type 2 H antigen, type 2 A antigen, type 2 B antigen, type 3 H antigen, type 3 A antigen, type 3 B antigen, type 4 H antigen, type 4 A antigen, type 4 B antigen, Gal-alpha 1-3Gal antigen, HNK-1, 3-O-sulfated Gal, Sd^{a} antigen, Forssman antigen, polysialic acid, P^{k} antigen, P antigen and P1 antigen. Antibodies are also available to glycosaminoglycans and to many glycolipid antigens, including globoside, GD3, GD2, GM2, GM1, asialo-GM1, GD1a, GD1b, GD3, and GQ1b. See Varki et al., Essentials of Glycobiology 4th ed. Cold Spring Harbor (NY): Cold Spring Harbor Laboratory Press (2022) Chapter 48, which is incorporated hercin by reference.

A carbohydrate binding reagent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

Particularly useful carbohydrate binding reagents recognize a monosaccharide moiety, disaccharide moiety, trisaccharide moiety or tetrasaccharide moiety. The trisaccharide moieties or tetrasaccharide moieties can be linear saccharide moieties. Alternatively, the trisaccharide moieties or tetrasaccharide moieties can be branched saccharide moieties. In some cases, carbohydrate binding reagents recognize moieties present at a glycan terminus, such as terminal monosaccharide, disaccharide or trisaccharide moieties. Some configurations of the compositions and methods set forth herein can employ a carbohydrate binding reagent that is relatively specific for a particular carbohydrate moiety. For example, carbohydrate binding reagents that are specific for moieties present at a glycan terminus can be used to distinguish glycans based on location of their epitopes at a glycan terminus from similar moieties that are present at internal positions of a glycan. Alternatively, carbohydrate binding reagents can be promiscuous, binding to a plurality of different glycans. Promiscuity can be due to an individual carbohydrate binding reagent recognizing an epitope that is present in multiple different glycans. For example, a carbohydrate binding reagent can be configured to recognize a carbohydrate epitope that is present in multiple different extant glycans in an array of different glycans. Promiscuity can also be due to a carbohydrate binding reagent recognizing two or more different epitopes. For example, a carbohydrate binding reagent can be configured to recognize two or more different carbohydrate epitopes present in different extant glycans in an array.

Another useful type of probe is a carbohydrate modifying reagent. For example, glycans can be labeled by reaction with N-azidoacetylgalactosamine, acetylated (Gal-NAz), N-azidoacetylglucosamine, acetylated (GlcNAz), and/or N-azidoacetylmannosamine, acctylated (ManNAz) followed by modified-Staudinger ligation with a labeled-phosphine reagent. Glycans can also be modified by incorporation of labeled saccharides using enzymes that participate in metabolic pathways for synthesizing and degrading glycans. Exemplary enzymes include N-glycosidases, such as peptide-N-glycosidase F, peptide-N-glycosidase A, peptide-N-glycosidase F-II, peptide-N-glycosidase H+, peptide-N-glycosidase Yl, or peptide-N-glycosidase Ar. Other exemplary glycans include endoglycosidases such as endoglycosidase A, endoglycosidase H, endoglycosidase M, endoglycosidase D, or endoglycosidase S. Labels can also be introduced using chemical reagents that react with glycans, examples of which include, hydrazine, ammonia, ammonium carbonate, N-bromosuccinimide, sodium hypochlorite, sodium hydroxide, or sodium borohydride. By way of further example, glycans can be labeled at their reducing ends by reductive amination. In a first step, a stable Schiff's base can be formed when the carbonyl carbon of an acyclic reducing sugar is attacked by a dye (e.g. anthranilic acid or 2-aminobenzamide) in a nucleophilic manner. Following the formation of the Schiff's base, the resulting imine group can be reduced using sodium cyanoborohydride to yield a stable labeled glycan. Fluorophores that are particularly useful for labeling glycans include, but are not limited to, anthranilic acid, 2-aminobenzamide, 2-aminopyridine, 2-aminoacridone, 7-Amino-1,3-naphthalenedisulfonic acid, 8-Aminonaphthalene-1,3,6-trisulfonic acid or 9-Aminopyrene-1,4,6-trisulfonic acid. These and other useful labelling reagents can be obtained from commercial suppliers such as MilliporeSigma (Burlington, MA) or Agilent (Santa Clara, CA).

A series of different probes that is contacted with glycans in a method set forth herein can include a plurality of different probes. The probes can differ with regard to the type of glycan recognized or the extent of reaction with different types of glycans. For example, carbohydrate binding reagents can differ with regard to the specificity with which epitopes are recognized. As such, different carbohydrate binding reagents can differ with regard to the carbohydrate moieties that they recognize. Alternatively or additionally, carbohydrate binding reagents can differ with regard to their strength of binding to particular epitopes. As such, different carbohydrate binding reagents can differ with regard to their equilibrium dissociation constant (K_{d}), equilibrium association constant (Kₐ), binding rate, binding rate constant (kₒₙ), dissociation rate, dissociation rate constant (k_{off}), binding probability, or avidity for a given glycan or moiety thereof.

A series of different probes can include at least 2, 5, 10, 25, 50, 100, 250, 500 or more different probes. Alternatively, a series of different probes can include at most 500, 250, 100, 50, 25, 10, 5 or 2 different probes. In some cases, a series of probes that is delivered to a glycan sample (e.g. an array of glycans) can include duplicate deliveries. For example, a given probe type can be delivered in at least 2, 3, 4, 5 or more probe delivery steps. A probe delivery step can be configured to deliver a single type of probe at a time such that only one probe type interacts with a glycan sample. Alternatively, probe delivery can result in multiple different probes being in simultaneous contact with a glycan sample. For example, at least 2, 3, 4, 5 or more different probes can be in simultaneous contact with an array of glycans or other glycan sample. It will be understood that probes need not be delivered serially in a method set forth herein. For example, a plurality of probes can be uniquely labeled to facilitate distinction of different probes that are simultaneously present in a glycan sample.

A method set forth herein can include a step of detecting positive recognition outcomes for interaction of a plurality of probes with extant glycans. Optionally, glycans can be detected at single-analyte resolution. In an exemplary configuration, single glycan resolution can be achieved by spatial or temporal separation of one glycan from another. In other configurations, single-glycoconjugate resolution is achieved by spatial or temporal separation of one glycoconjugate from another. In the latter configuration, multiple glycans that are attached to a given glycoconjugate need not be resolved from each other. Rather, the glycoconjugate is detected as a collection of multiple glycans. Spatial separation of glycans or glycoconjugates can be achieved by attachment of individual glycans to respective addresses of an array or by attachment of individual glycoconjugates to respective addresses of an array. Alternatively to single-analyte resolution, a detection method can be carried out at ensemble-resolution or bulk-resolution. Bulk-resolution configurations acquire a composite signal from a plurality of glycans or glycoconjugates. A composite signal can be acquired from a population of multiple sets of glycans or glycoconjugates, for example, in a well or cuvette, or on a solid support surface.

Detection of multiple glycan or glycoconjugates can be performed in a multiplex format. In multiplexed formats, different glycans or glycoconjugates can be attached to different unique identifiers (e.g. sites in an array), and the sets can be manipulated and detected in parallel. For example, a fluid containing one or more different probes can be delivered to an array of addresses, each address having an immobilized glycan or glycoconjugate, such that the addresses of the array are in simultaneous contact with the probe(s). The glycans or glycoconjugates can be attached to the respective addresses via particles or, alternatively, the glycans or glycoconjugates can be directly attached to the respective addresses. Moreover, a plurality of addresses can be observed in parallel allowing for rapid detection of probe interaction events.

A glycan or glycoconjugate can be attached to a unique identifier using any of a variety of means. Exemplary attachments include, but are not limited to, covalent or non-covalent attachments. Exemplary reagents and methods that can be used to attach glycans or glycoconjugates to solid supports are set forth in US Pat. App. Pub. No. 2021/0101930 A1 or U.S. patent application Ser. No. 17/692,035, each of which is incorporated herein by reference. Optionally, a glycan or glycoconjugate can be attached to a particle and the particle can be attached to an address on a solid support or to another unique identifier.

Interaction of probes with glycans or glycoconjugates can be detected using any of a variety of techniques that are appropriate to the assay components used. For example, binding of carbohydrate binding reagents can be detected by acquiring a signal from a label attached to a carbohydrate binding reagent when bound to a glycan or glycoconjugate, acquiring a signal from a label attached to a glycan or glycoconjugate when bound to an affinity reagent, or acquiring signal(s) from labels attached to a carbohydrate binding reagent and a glycan or glycoconjugate to which the carbohydrate binding reagent is bound (e.g. signals produced via fluorescence resonance transfer between a label on the carbohydrate binding reagent and a label on a glycan or glycoconjugate). In some configurations, a complex between a carbohydrate binding reagent and a glycan or glycoconjugate need not be directly detected, for example, in formats where a nucleic acid tag or other moiety is created or modified as a result of binding. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or US Pat. App. Pub. No. 2022/0162684 A1, each of which is incorporated herein by reference.

A plurality of glycans or glycoconjugates can be detected by obtaining multiple separate and non-identical measurements of the plurality. In particular configurations, the individual measurements may not, by themselves, be sufficiently accurate or specific to produce a specific characterization, but an aggregation of the multiple non-identical measurements can allow a characterization to be made with a high degree of specificity using a decoding method. For example, the multiple separate measurements can include subjecting the plurality of glycans or glycoconjugates to probes that are promiscuous with regard to recognizing multiple different glycans suspected of being present in a given sample from which the plurality is derived. For example, a first measurement carried out using a first promiscuous probe may perceive a first subgroup of glycans or glycoconjugates without differentiating the identity of one glycan or glycoconjugate in the subgroup from another glycan or glycoconjugate in the subgroup. A second measurement carried out using a second promiscuous probe may perceive a second subgroup of glycans or glycoconjugates, again, without differentiating the identity of one glycan or glycoconjugate in the second subgroup from another glycan or glycoconjugate in the second subgroup. However, a comparison of the first and second measurements can distinguish one glycan or glycoconjugate from others.

In particular configurations, a plurality of glycans or glycoconjugates can be detected using one or more carbohydrate binding reagents having known or measurable binding affinity for the glycans or glycoconjugates. For example, a carbohydrate binding reagent can bind a glycan or glycoconjugate to form a complex with at least one of the glycans or glycoconjugates, and a signal produced by the complex can be detected. Although performing a single reaction between a promiscuous probe and a plurality of glycans or glycoconjugates may yield ambiguous results regarding the identity of the different glycans or glycoconjugates to which it binds, the ambiguity can be resolved when the results are combined with other identifying information in a decoding method. For example, a plurality of different promiscuous probes can be contacted with glycans or glycoconjugates derived from a complex sample, wherein the plurality or probes is configured to produce a different outcome profile for each candidate glycan or glycoconjugate suspected of being present in the sample. In this example, each of the probes is distinguishable from the other probes, for example, in the case of probes that are carbohydrate binding reagents, due to unique labeling (e.g. different reagents have different luminophore labels), unique spatial location (e.g. different reagents are located at different sites in an array), and/or unique time of use (e.g. different reagents are delivered in series to a population of glycans or glycoconjugates).

A method or system of the present disclosure can utilize a database including a set of candidate glycans or glycoconjugates. The database can include, for each candidate glycan or glycoconjugate, the probability of a positive recognition outcome for the glycan or glycoconjugate with each probe selected among a plurality of different probes. For example, a database can include, for each candidate glycan or glycoconjugate, the probability of a positive binding outcome for the glycan or glycoconjugate with each carbohydrate binding reagent selected among a plurality of different carbohydrate binding reagents. In some configurations, a database can include, for each candidate glycan or glycoconjugate, the probability of a negative recognition outcome for the glycan or glycoconjugate with each probe selected among a plurality of different probes. For example, a database can include, for each candidate glycan or glycoconjugate, the probability of a negative binding outcome for the glycan or glycoconjugate with each carbohydrate binding reagent selected among a plurality of different carbohydrate binding reagents.

A plurality of candidate glycans or glycoconjugates may include at least 10, 25, 50, 75, 100, 500, 1×10³, 1×10⁶, or more different candidate glycans or glycoconjugates. In some cases, a complete glycoproteome or substantial fraction thereof can be included. For example, a database can include at least 10%, 25%, 50%, 75%, 90%, 95%, 99% or more of the glycoproteins known, or suspected, to be present in a proteome. A database may include candidate glycans or glycoconjugates from a single organism or from more than one organism. For example, a database can include glycans or glycoconjugates from a given ecosystem such as a microbiome or environmental sample; from a particular family, class or genera of species; or all known glycans or glycoconjugates from all known species.

Information that can be included in a database of candidate glycans or glycoconjugates includes, for example, chemical structures. Optionally, information other than glycan structures can be included in a database. Particularly useful information that can be included in a database includes, for example, binding characteristics for binding of one or more carbohydrate binding reagents to a glycan or glycoconjugate. However, such information need not be included in the database and can instead be provided by a trained model. For example, the information can include a probability for each of a plurality of probes interacting with each of a plurality of candidate glycans or glycoconjugates. In some configurations, such probabilities or other characteristics are derived empirically, for example, from training assays carried out between known probes and known glycans or glycoconjugates. In some embodiments, probabilities or other characteristics are derived based on a priori information such as presence of a suspected epitope in the structure of a candidate glycan or glycoconjugate.

A database can include a probability or likelihood that a candidate glycan or glycoconjugate would generate a particular positive recognition outcome with one or more probes. A database can further include a probability or likelihood that a candidate glycan or glycoconjugate would generate a negative recognition outcome with one or more probes.

The present disclosure provides methods that can provide accurate identification of glycans or glycoconjugates despite ambiguities and imperfections that arise in many contexts. In some configurations, methods for identifying, quantitating or otherwise characterizing glycans or glycoconjugates in a sample utilize a trained model that evaluates the likelihood or probability that one or more candidate glycans or glycoconjugates will have produced an empirically observed outcome profile. The trained model can include information regarding expected recognition outcomes (e.g. binding or non-binding) for interaction of one or more probes with one or more candidate glycans or glycoconjugates. The information can include a priori characteristics of a glycan or glycoconjugate such as the carbohydrate composition of the glycan or glycoconjugate or the nature of bonds between monosaccharide moieties in the glycan or glycoconjugate.

A decoding method can be configured to evaluate the degree of compatibility of one or more empirical outcome profiles (e.g. obtained from one or more extant glycans or glycoconjugates) with results computed for various candidate glycans or glycoconjugates using a trained model. For example, to identify an unknown glycan in a sample of many glycans, an empirical binding profile for an extant glycan can be compared to results computed from the trained model for many or all candidate glycans suspected of being in the sample. In some configurations of the methods set forth herein, identity for the unknown glycan is determined based on a likelihood of the extant glycan being a particular candidate glycan given the empirical outcome profile, or based on the probability of a particular candidate glycan generating the empirical outcome profile. Methods for identifying polypeptides using promiscuous reagents, serial binding measurements and/or decoding with trained models are set forth, for example, in U.S. Pat. No. 10,473,654 US Pat. App. Pub. No. 2020/0318101 A1 or Egertson et al., BioRxiv (2021), DOI: 10.1101/2021.10.11.463967, each of which is incorporated herein by reference. The methods can be modified for use in characterizing glycans or glycoconjugates in accordance with the teachings set forth herein.

A method of the present disclosure can include a step of identifying an extant glycan or glycoconjugate from results of a probe interaction assay. The identification can include determining the structure of a glycan, for example, based on the combination of epitopes detected in an extant glycan or glycoconjugate. The structures for a plurality of candidate glycans can be evaluated for the presence of the combination of detected epitopes. Optionally, the structures for a plurality of candidate glycans can also be evaluated for absence of epitopes that were determined not to be present in the extant glycan or glycoconjugate.

A glycan or glycoconjugate can be identified at varying degrees of specificity using methods set forth herein. For example, a glycan or glycoconjugate can be identified at a very high level of specificity wherein the structure of the glycan or glycoconjugate becomes known. Alternatively, a glycan or glycoconjugate can be identified at a level of specificity that does not necessarily reveal the complete structure of the glycan or glycoconjugate. Nevertheless, the results of a method set forth herein may provide a unique signature for a glycan or glycoconjugate that can be correlated with a characteristic of a sample from which the glycan or glycoconjugate was derived. The signature can optionally be used as a basis for diagnosis, prognosis, or other characterization of samples from which the glycan or glycoconjugate is derived. In some cases, a signature can include detected characteristics of a plurality of glycans or glycoconjugates. Such a signature can correlate the presence or absence of a plurality of molecular species with a phenotype or other characteristic of a biological sample.

Optionally decoding can include determining the most probable candidate glycan or the most probable group of candidate glycans corresponding to different extant glycans in a sample. Similarly, decoding can include determining the most probable candidate glycoconjugate or the most probable group of candidate glycoconjugates corresponding to different extant glycoconjugates in a sample. Decoding can be carried out by a computer. The computer can utilize a database of candidate glycans or glycoconjugates when performing the decoding step. The computer can optionally use a machine learning algorithm to determine the candidate glycans or glycoconjugates in a database corresponding to different extant glycans or glycoconjugates detected in a sample. Examples of machine learning algorithms that can be useful include support vector machines (SVMs), neural networks, convolutional neural networks (CNNs), deep neural networks, cascading neural networks, k-Nearest Neighbor (k-NN) classification, random forests (RFs), and other types of classification and regression trees (CARTs). Other algorithms that can be employed for decoding include, but are not limited to deep learning, statistical learning, supervised learning, unsupervised learning, clustering, expectation maximization, maximum likelihood estimation, Bayesian inference, linear regression, logistic regression, binary classification, multinomial classification, or other pattern recognition algorithm.

A decoding step can be carried out by calculating, for an extant glycan or glycoconjugate, a probability that an outcome profile is observed given that a candidate glycan or glycoconjugate in a database is the extant glycan or glycoconjugate. Looking to the example of decoding an array of glycans or glycoconjugates, decoding can be carried out by calculating, for an individual address of the array, a probability that a particular outcome profile is observed given that a particular candidate glycan or glycoconjugate in the database is present at the individual address. The calculation for the individual address can be performed for a plurality of candidate glycans or glycoconjugates in the database. A candidate glycan or glycoconjugate having a candidate outcome profile that is most consistent with the empirically observed outcome profile for the individual address can be identified as the most likely identity for the extant glycan or glycoconjugate at the individual address. Similar calculations can be performed for respective addresses in the array. In some cases, a single candidate glycan or glycoconjugate can be identified for an individual address. Alternatively, decoding can determine a probability or likelihood that more than one candidate glycan or glycoconjugate in a database would generate one or more of the recognition outcomes for a given address of an array of glycans and glycoconjugates.

The present disclosure provides a decoding method or algorithm that can be used to evaluate the results of a glycan assay. The results can be used to identify or otherwise characterize glycans or glycoconjugates. In some configurations, distinct and reproducible outcome profiles may be observed for some or even a substantial majority of glycans or glycoconjugates that are to be identified in a sample. However, in many cases one or more probe reactions produces inconclusive or even aberrant results and this, in turn, can yield ambiguous outcome profiles. For example, observation of binding outcomes at single-molecule resolution can be particularly prone to ambiguities due to stochasticity in the binding behavior of glycans or glycoproteins when observed individually. The present disclosure provides decoding methods that can provide accurate identification of glycans or glycoconjugates despite ambiguities and imperfections that can arise in single-molecule formats or in other contexts.

In some configurations, methods for identifying or characterizing one or more extant glycans or glycoconjugates in a sample utilize a decoding method that analyzes an empirical outcome profile acquired for a plurality of probe interactions observed between each extant glycan or glycoconjugate in a sample and a plurality of probes, and then the empirical outcome profile is evaluated with respect to the reactivity of the probes with a plurality of candidate glycans or glycoconjugates. The plurality of candidate glycans or glycoconjugates can include species that are known or suspected of being present in the sample. The decoding algorithm can output the identity of a given extant glycan or glycoconjugate as the candidate glycan or glycoconjugate that has recognition outcomes that are most compatible with the empirically observed recognition outcomes. This compatibility can be determined based on a trained model that represents the recognition of each of the candidate glycans or glycoconjugates by each of the probes that were used to produce the empirical outcome profile. A strong candidate glycan or glycoconjugate can be identified as one for which the modeled recognition outcomes are more consistent with the empirical outcome profile as compared to the other candidate glycans or glycoconjugates evaluated.

A decoding method of the present disclosure can be configured to evaluate positive recognition outcomes. For example, a decoding method can evaluate positive recognition outcomes without evaluating negative binding outcomes. Alternatively, a strong candidate glycan or glycoconjugate can be identified as one for which a combination of positive recognition outcomes and negative recognition outcomes is more consistent with the empirical outcome profile as compared to the other candidate glycans or glycoconjugates evaluated. A candidate glycan or glycoconjugate can be identified as a weak identity or even incorrect identity based on having many instances where positive recognition outcomes and/or negative recognition outcomes are inconsistent with the empirical outcome profile being evaluated. The strongest candidate glycan or glycoconjugate can be deemed the most likely identity for the extant glycan or glycoconjugate. Confidence in this identification can be computed as a relative measure of the compatibility of the most likely glycan or glycoconjugate compared to all of the other candidate glycans or glycoconjugates.

A computer processor can be configured to execute a decoding method that outputs identities for one or more extant glycans or glycoconjugates based on various inputs. A particularly useful input is empirical data for binding of an extant glycan or glycoconjugate to a plurality of different carbohydrate binding reagents. The binding data can be in the form of an empirical binding profile that includes a plurality of binding outcomes. An empirical binding profile can include positive binding outcomes or negative binding outcomes. The same can be true for a candidate outcome profile.

An empirical outcome profile can be input to a decoding method set forth herein. For example, the empirical outcome profile can be input to a computer processor that performs the decoding method. A series of empirical recognition outcomes that constitute an empirical outcome profile can be acquired using probe reactions such as those set forth herein or known in the art. Alternatively, an outcome profile can be obtained from a simulation and used similarly to an empirical outcome profile. Each empirical recognition outcome in an outcome profile can result from one probe reaction among a plurality of probe reactions carried out between a plurality of probes and a particular extant glycan or glycoconjugate. An empirical outcome profile can be decoded after all recognition outcomes have been acquired for a given extant glycan or glycoconjugate. Alternatively, for example, when recognition outcomes are acquired serially, decoding can occur in real time such that evaluation of an empirical recognition outcome from an earlier probe reaction in the series is initiated, and perhaps completed, prior to, or during, acquisition of an empirical recognition outcome for a subsequent probe reaction in the series. A plurality of empirical recognition outcomes need not necessarily be acquired serially, for example, instead being acquired such that some or all recognition outcomes in an empirical outcome profile are acquired from probe reactions that occur in parallel.

A trained model can be input to a decoding method set forth herein. For example, the trained model can be input to a computer processor that performs the decoding method. Optionally, a trained model can include a function for determining probability of a specific interaction occurring between a glycan or glycoconjugate and each of a plurality of probes. In some configurations, a trained model can include a function for determining probability of a specific binding event occurring between a glycan or glycoconjugate and each of a plurality of carbohydrate binding reagents. Epitopes evaluated by the model can have any of a variety of characteristics of interest. For example, the epitopes can have a defined length (e.g. the epitope length being less than or equal to 2, 3, 4, 5 or 6 monosaccharide subunits) or chemical composition (e.g. types of saccharides or linkages between saccharides).

In some configurations of methods set forth herein, the number of identifications generated for each unique candidate glycan or glycoconjugate is used to determine the quantity of each candidate glycan or glycoconjugate in a sample. In some configurations, a collection of glycan or glycoconjugate identifications and associated probabilities can be filtered to only contain identifications of a high score, high confidence, and/or low false discovery rate.

A decoding method can output information pertaining to the identity or quantity for one or more extant glycans or glycoconjugates. The information output for a given glycan or glycoconjugate can be in the form of a determined identity for the protein or in the form of a probability or likelihood for one or more identity of the glycan or glycoconjugate. For example, the most likely identity for an extant glycan or glycoconjugate, the likelihood or probability of the extant glycan or glycoconjugate having a particular identity, or both can be output by a decoding method. A decoding method can output a non-digital or non-binary score for the identity of a given extant glycan or glycoconjugate or for the likelihood of the extant glycan or glycoconjugate having a particular identity. For example, probability or likelihood scores can be output in the form of a continuous value between 0 and 1, or percent value between 0% and 100%. In some configurations, a digital or binary score that indicates one of two discrete states can be output to indicate the identity of a glycan or glycoconjugate.

In some configurations of the methods set forth herein, a paper or electronic report can be output from a computer, the report identifying one or more glycans or glycoconjugates in an assay or sample from which the glycans or glycoconjugates were obtained. The report may further indicate, for each of the candidate glycans or glycoconjugates, a confidence level for the candidate glycan or glycoconjugate being present in the sample. The confidence level may comprise a probability or likelihood value. Alternatively, the confidence level may include a probability value with an error value. Alternatively, the confidence level may include a range of probability values, optionally with a confidence (e.g., at least about 0.8, 0.9, 0.99, 0.999 or higher confidence on a scale of 0 to 1). A report may further indicate a list of candidate glycans or glycoconjugates identified below an expected false discovery rate threshold (e.g., a false discovery rate below 0.2, 0.1, 0.05, 0.01, 0.001 or lower on a scale of 0 to 1).

A decoding algorithm or model can be trained using data from probe interaction assays with known glycans or glycoconjugates. For example, an assay can be performed using an array having known glycans or glycoconjugates at known addresses. The results of interacting known probes with the addresses, for example the results of binding known carbohydrate binding reagents with the addresses, can be evaluated and used for training. The probes, glycans, glycoconjugates, arrays and assays can be configured as set forth herein and using known components.

A method of the present disclosure can be configured to include a step of modifying one or more glycan and then performing steps to detect the glycan. In some cases, an assay can be configured to detect a glycan before and after contacting the glycan with a glycan modifying reagent. Changes in the detectability of the glycan can indicate a property of the glycan, such as the presence of a bond (e.g. N-glycosyl linkage or O-glycosyl linkage). Conversely, similar or substantially unchanged detectability before and after subjecting a glycan to a glycan modifying reagent can indicate absence of a bond.

Accordingly, a method of characterizing glycans can include steps of (a) providing an array of extant glycans, wherein the array includes a plurality of addresses, wherein different extant glycans are attached to different addresses of the array; (b) contacting the array with a plurality of different probes, the different probes recognizing different carbohydrate moieties; (c) detecting positive recognition outcomes of the plurality of different probes at individual addresses of the array, thereby producing outcome profiles for the addresses; (d) contacting the array with a glycan modifying reagent; (c) contacting the array with a second plurality of different probes, the different probes recognizing different carbohydrate moieties; (f) detecting positive recognition outcomes of the second plurality of different probes at individual addresses of the array, thereby producing second outcome profiles for the addresses; (g) providing a database including a set of candidate glycans, the database including, for each candidate glycan, the probability of a positive recognition outcome for the plurality of different probes; and (h) determining with a computer, using the database and the outcome profiles, candidate glycans in the database corresponding to different extant glycans in the array.

Any of a variety of glycan modifying reagents can be used. For example, an N-glycosidase can be used. Detection of a glycan before and after contact with an N-glycosidase can indicate the presence or absence of an N-glycosyl linkage in a glycan. Exemplary N-glycosidases include, but are not limited to, peptide-N-glycosidase F, peptide-N-glycosidase A, peptide-N-glycosidase F-II, peptide-N-glycosidase H+, peptide-N-glycosidase Yl, or peptide-N-glycosidase Ar. Another useful type of glycan modifying reagent is an endoglycosidase. An endoglycosidase can release oligosaccharide moieties from glycans by cleaving bonds between saccharides that are not terminal saccharides in the glycan. Exemplary endoglycosidases include, but are not limited to endoglycosidase A, endoglycosidase H, endoglycosidase M, endoglycosidase D, or endoglycosidase S. Another useful type of glycan modifying reagent is a chemical modifying reagent such as hydrazine, ammonia, ammonium carbonate, N-bromosuccinimide, sodium hypochlorite, sodium hydroxide, or sodium borohydride. Glycan can be modified with azides and the azides can be subsequently subjected to a click chemistry reaction (e.g. copper catalyzed azide-alkyne cycloaddition or copper-free reactions between azide and ring strained alkynes) to attach moieties of interest, such as luminophores or other labels. See, for example, Baskin et al., Proc. Nat'l Acad. Sci. USA 104:16793-16797 (2007), which is incorporated herein by reference.

A method of the present disclosure can be configured to include a step of modifying one or more glycoconjugate and then performing steps to detect the glycan. In some cases, an assay can be configured to detect a glycoconjugate before and after contacting the glycan with a glycan modifying reagent. Similarly, a glycoconjugate having at least one glycan moiety attached to a non-glycan moiety can be modified at the non-glycan moiety. Changes in the detectability of the glycoconjugate can indicate a property of the glycoconjugate. For example, a glycoprotein can be treated with a protease, a glycan removing enzyme, a glycan adding enzyme, or chemical reagent that modifies particular amino acid side chains. Changes in detectability of the glycoprotein can indicate structural characteristics or even the identity of the glycoprotein.

Accordingly, a method of characterizing glycoconjugates can include steps of (a) providing an array of extant glycoconjugates, wherein the array includes a plurality of addresses, wherein different extant glycoconjugates are attached to different addresses of the array; (b) contacting the array with a plurality of different probes, the different probes recognizing different moieties of the glycoconjugate; (c) detecting positive recognition outcomes of the plurality of different probes at individual addresses of the array, thereby producing outcome profiles for the addresses; (d) contacting the array with a glycoconjugate modifying reagent; (e) contacting the array with a second plurality of different probes, the different probes recognizing different glycoconjugate moieties; (f) detecting positive recognition outcomes of the second plurality of different probes at individual addresses of the array, thereby producing second outcome profiles for the addresses; (g) providing a database including a set of candidate glycoconjugates, the database including, for each candidate glycoconjugate, the probability of a positive recognition outcome for the plurality of different probes; and (h) determining with a computer, using the database and the outcome profiles, candidate glycoconjugates in the database corresponding to different extant glycoconjugates in the array.

A method that is configured to characterize glycans or glycoconjugates can include multiple iterations of the multi-step process of (i) detecting a glycan (e.g. a glycan molecule or glycan moiety of a glycoconjugate), (ii) then contacting the glycan with a glycan modifying reagent, and (iii) then detecting the glycan or glycoconjugate to determine presence or absence of a change in the glycan structure. Such an approach can be particularly useful when evaluating glycans having complex structures. For example, a variety of lectins and other carbohydrate binding reagents are specific for carbohydrate moieties located at peripheral or terminal positions of a glycan. Repeated steps of modification (e.g. trimming saccharide units from a glycan) and detecting can allow the structure of a glycan to be elucidated based on knowledge of the specificity of the modification reagent (e.g. known structure for a linkage that is cleaved), increased signal from a given carbohydrate binding reagent due to the modification exposing an epitope that had been inaccessible in a previous detection step using the given carbohydrate binding reagent, and/or decreased signal from a given carbohydrate binding reagent due to the modification removing an epitope that had been bound by a given carbohydrate binding reagent in a previous detection step using the carbohydrate binding reagent.

A method of the present disclosure can include steps of detecting at least one non-glycan moiety of a glycoconjugate. For example, the protein moiety of a glycoprotein can be detected using any of a variety of assays designed for detecting proteins. For example, a protein can be detected using one or more affinity reagents having binding affinity for amino acid motifs that function as epitopes for the affinity reagents. The affinity reagent and the protein can bind each other to form a complex and, during or after formation, the complex can be detected. The complex can be detected directly, for example, due to a label that is present on the affinity reagent or protein. In some configurations, the complex need not be directly detected, for example, in formats where the complex is formed and then the affinity reagent, protein, or a label component that was present in the complex is subsequently detected.

Accordingly, the present disclosure provides is a method of characterizing glycoproteins. The method can include steps of (a) providing an array of extant glycoproteins, wherein the array includes a plurality of addresses, wherein different extant glycoproteins are attached to different addresses of the array; (b) contacting the array with a plurality of different probes; (c) detecting positive recognition outcomes for the plurality of different probes at individual addresses of the array, thereby producing outcome profiles for the addresses; (d) providing a database including a set of candidate glycans or glycoproteins, the database including, for each candidate glycan or glycoprotein, the probability of a positive recognition outcome for the plurality of different probes; (c) determining with a computer, using the database and the outcome profiles, candidate glycans or glycoproteins in the database corresponding to glycoproteins at addresses of the array; (f) contacting the array with a plurality of different affinity reagents, wherein the affinity reagents recognize epitopes including amino acids; (g) detecting positive binding outcomes for the plurality of different affinity reagents at individual addresses of the array, thereby producing protein outcome profiles for the addresses; (h) providing a database comprising a set of candidate proteins, the database including, for each candidate protein, the probability of a positive binding outcome for the plurality of different affinity reagents; and (i) determining with a computer, using the database and the protein outcome profiles, candidate proteins in the database corresponding to proteins at addresses of the array.

A method of characterizing glycoproteins can include steps of detecting the glycan moieties of the glycoproteins and steps of detecting the protein moieties of the glycoproteins. In some configurations the glycan moieties are detected prior to detecting the protein moieties. For example, steps (b) and (c) of the above method can be carried out prior to steps (f) and (g). Alternatively, the protein moieties are detected prior to detecting the glycan moieties. For example, steps (b) and (c) of the above method can be carried out after steps (f) and (g).

Optionally, a method of detecting glycoproteins can include a step of contacting the glycoproteins with a glycosidase or chemical reagent, thereby removing glycan moieties from protein moieties of the glycoproteins. For example, a glycan removal step can be carried out prior to step (f) of the above method. Alternatively or additionally, the glycan removal step can be carried out after step (b) and/or step (c) of the above method.

Many protein assays, such as enzyme linked immunosorbent assay (ELISA), achieve high-confidence characterization of one or more proteins in a sample by exploiting high specificity binding of affinity reagents to the protein(s) and detecting the binding event while ignoring all other proteins in the sample. Binding assays can be carried out by detecting immobilized affinity reagents and/or immobilized proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection assays employ SOMAmer reagents and SOMAscan assays commercialized by Soma Logic (Boulder, CO). In one configuration, a sample is contacted with aptamers that are capable of binding proteins with specificity for amino acid sequences of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

Turning to the example of an array-based assay, the identity of an extant protein (e.g. the protein moiety of a glycoprotein) at any given address is typically not known prior to performing the assay. An assay can be used to identify proteins at one or more addresses in the array. A plurality of affinity reagents, optionally labeled (e.g. with fluorophores), can be contacted with the array, and the presence of affinity reagents can be detected from individual addresses to determine binding outcomes. A plurality of different affinity reagents can be delivered to the array and detected serially, such that each cycle detects binding outcomes for an individual affinity reagent. In some configurations, a plurality of affinity reagents can be detected in parallel, for example, when different affinity reagents are distinguishably labeled.

In particular configurations, the methods can be used to identify a number of different proteins that exceeds the number of affinity reagents used. For example, the number of proteins identified can be at least 5×, 10×, 25×, 50×, 100× or more than the number of affinity reagents used. This can be achieved, for example, by (1) using promiscuous affinity reagents that bind to multiple different proteins suspected of being present in a given sample, and (2) subjecting the protein sample to a set of promiscuous affinity reagents that, taken as a whole, are expected to bind each protein in a different combination, such that each protein is expected to generate a unique profile of binding and non-binding events. Promiscuity of an affinity reagent can arise due to the affinity reagent recognizing an epitope that is known to be present in a plurality of different proteins. For example, epitopes having relatively short amino acid lengths such as dimers, trimers, tetramers or pentamers can be expected to occur in a substantial number of different proteins in a typical proteome. Alternatively or additionally, a promiscuous affinity reagent may recognize different epitopes (e.g. epitopes differing from each other with regard to amino acid composition or sequence). For example, a promiscuous affinity reagent that is designed or selected for its affinity toward a first trimer epitope may bind to a second epitope that has a different sequence of amino acids compared to the first epitope.

Although performing a single binding reaction between a promiscuous affinity reagent and a complex protein sample may yield ambiguous results regarding the identity of the different proteins to which it binds, the ambiguity can be resolved by decoding the binding profiles for each protein using machine learning or artificial intelligence algorithms that are based on probabilities for the affinity reagents binding to candidate proteins. For example, a plurality of different promiscuous affinity reagents can be contacted with a complex population of proteins, wherein the plurality is configured to produce a different binding profile for each candidate protein suspected of being present in the population. The plurality of promiscuous affinity reagents can produce a binding profile for each individual protein that can be decoded to identify a unique combination of positive (i.e. observed binding events) and/or negative binding outcomes (i.e. observed non-binding events), and this can in turn be used to identify the individual protein as a particular candidate protein having a high likelihood of exhibiting a similar binding profile.

Binding profiles can be obtained for each protein and decoded. In many cases one or more binding events produces inconclusive or even aberrant results and this, in turn, can yield ambiguous binding profiles. For example, observation of a binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include information regarding the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an unknown protein in a sample, an empirical binding profile for the protein can be compared to results computed by the binding model for many or all candidate proteins suspected of being in the sample. A machine learning or artificial intelligence algorithm can be used. An algorithm used for decoding can utilize Bayesian inference or other machine learning algorithms set forth herein or known in the art. In some configurations, identity for an unknown protein is determined based on a likelihood of the unknown protein being a particular candidate protein given the empirical binding pattern or based on the probability of a particular candidate protein generating the empirical binding pattern. Particularly useful decoding methods are set forth, for example, in U.S. Pat. No. 10,473,654; US Pat. App. Pub. No. 2020/0318101 A1; U.S. patent application Ser. No. 18/045,036, or Egertson et al., BioRxiv (2021), DOI: 10.1101/2021.10.11.463967, each of which is incorporated herein by reference.

One or more compositions set forth herein can be provided in kit form including, if desired, a suitable packaging material. In one configuration, for example, a particle, solid support, flow cell, array, probe, carbohydrate binding reagent, affinity reagent, carbohydrate modifying reagent and/or other composition set forth herein can be provided in one or more vessels. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, mylar, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular polypeptide or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection system. For example, the cartridge can be engaged with a detection system such that contents of the cartridge are in fluidic communication with a detection apparatus of the system or with a flow cell engaged with the detection apparatus. A cartridge can be engaged with a detection apparatus such that contents of the cartridge can be observed by the detection apparatus, for example, using an assay set forth herein.

One or more steps of a method set forth herein can be carried out in a detection system. Accordingly, a detection system can be configured to execute one or more steps of a method set forth herein. For example, a detection system can be configured to execute one or more steps of a method for characterizing glycans. A method set forth herein can be configured to improve the accuracy of the detection system. For example, the detection system can provide an initial identity or characterization for one or more glycans and a decoding method set forth herein can be used to output a subsequent identity or characterization that is more accurate or otherwise improved compared to the initial identity or characterization. A detection system can be composed of one or more apparatus. For example, a single apparatus can include some or all components exemplified herein. Alternatively, a system can include a combination of multiple apparatus that are optionally networked or otherwise connected to each other.

The present disclosure provides a system for detecting glycans. The system can include (a) a detector configured to acquire signals from a plurality of interactions occurring between a plurality of different probes and a plurality of different extant glycans in a sample; (b) a database including information characterizing or identifying a plurality of candidate glycans; (c) a computer processor configured to: (i) communicate with the database, (ii) process the signals to produce a plurality of outcome profiles, wherein each of the outcome profiles includes a plurality of recognition outcomes for interaction of an extant glycan of (a) to the plurality of different probes, wherein individual recognition outcomes of the plurality of recognition outcomes include a measure of interaction between an extant glycan of (a) and a different probe of the plurality of different probes, (iii) process the recognition profiles to determine a probability for each of the probes interacting with each of the candidate glycans in the database according to an interaction model for each of the probes; and (iv) output an identification of selected candidate glycans, the selected candidate glycans being candidate glycans in the database having a probability for interaction with each of the probes that is most compatible with the plurality of recognition outcomes for the extant glycans.

The present disclosure further provides a system for detecting glycoproteins. The system can include (a) a detector configured to acquire signals from a plurality of interactions occurring between a plurality of different probes and a plurality of different extant glycoproteins in a sample; (b) a database including information characterizing or identifying a plurality of candidate glycans or glycoproteins; (c) a computer processor configured to: (i) communicate with the database, (ii) process the signals to produce a plurality of outcome profiles, wherein each of the outcome profiles includes a plurality of recognition outcomes for interaction of an extant glycoprotein of (a) to the plurality of different probes, wherein individual recognition outcomes of the plurality of recognition outcomes include a measure of interaction between an extant glycoprotein of (a) and a different probe of the plurality of different probes, (iii) process the outcome profiles to determine a probability for each of the probes interacting each of the candidate glycans in the database according to a trained model for each of the probes; and (iv) output an identification of selected candidate glycans or glycoproteins, the selected candidate glycans or glycoproteins being a candidate glycans or glycoproteins in the database having a probability for interaction with each of the probes that is most compatible with the plurality of recognition outcomes for the extant glycans or glycoproteins.

Optionally, the above system can further include a database having information characterizing or identifying a plurality of candidate proteins. The detector can be further configured to acquire signals from a plurality of reactions occurring between a plurality of different affinity reagents and the plurality of different extant glycoproteins in the sample. As a further option, the computer processor of the system can be further configured to (v) process the signals to produce a plurality of outcome profiles, wherein each of the outcome profiles includes a plurality of binding outcomes for binding of an extant glycoprotein of (a) to the plurality of different affinity reagents, wherein individual binding outcomes of the plurality of binding outcomes include a measure of binding between an extant glycoprotein of (a) and a different affinity reagent of the plurality of different affinity reagents, and (vi) process the binding profiles to determine a probability for each of the affinity reagents interacting with each of the candidate proteins in the database according to a binding model for each of the affinity reagents. Optionally, the computer processor can be further configured to output an identification of selected candidate proteins, the selected candidate proteins being candidate proteins in the database having a probability for interaction with each of the affinity reagents that is most compatible with the plurality of binding outcomes for the extant glycoproteins.

A system of the present disclosure can include: (a) a detector configured to detect recognition outcomes for interactions of a plurality of probes with an array of addresses, each of the addresses having an extant glycan of a plurality of different extant glycans; (b) a database including a plurality of candidate glycans; and (c) a computer processor configured to analyze the recognition outcomes and the candidate glycans to characterize the extant glycans. Optionally, the computer processor is configured to output an identity or other characteristic for the extant glycans.

A system can include a detector, such as those known in the art for detecting a label or analyte set forth herein. A detector can be configured to collect signals (e.g. optical signals) from an array or other vessel containing glycans, glycoproteins or other analytes. A camera such as a complementary metal-oxide-semiconductor (CMOS) or charge-coupled device (CCD) camera can be particularly useful, for example, to detect optical labels such as luminophores. The detection system can further include an excitation source configured to excite extant glycans, glycoproteins or probes, for example, in an array or other vessel. A detection system can include a scanning mechanism configured to effect relative movement between a detector and an array or other vessel containing extant glycans or glycoproteins. Optionally, the scanning mechanism can be configured for time-delayed integration. Detectors that are capable of resolving analytes on an array surface including, for example, at single-molecule resolution can be particularly useful. Detectors used in DNA sequencing systems can be modified for use in a detection system or other apparatus set forth herein. Exemplary detectors are described, for example, in U.S. Pat. Nos. 7,057,026; 7,329,492; 7,211,414; 7,315,019 or 7,405,281, or US Pat. App. Pub. No. 2008/0108082 A1, each of which is incorporated herein by reference.

A system can further include a fluidics apparatus configured to contact components for a reaction or other step of a method set forth herein. In particular embodiments, reactions occur on arrays. Any of a variety of arrays can be present in the system, such as an array set forth herein. Glycans that are to be detected, for example those attached to an array, can be housed in any of a variety of reaction vessels. A particularly useful reaction vessel is a flow cell. A flow cell or other vessel can be present in a system in a permanent manner or in a removable manner, for example, being removable by hand or without the use of an auxiliary tool. A flow cell or other vessel that is present in a system can have a detection window through which a detector observes one or more glycans (e.g. an array of glycoproteins) or other analytes on the array. For example, an optically transparent window can be used in conjunction with an optical detector such as a fluorimeter or luminescence detector.

A fluidic apparatus can include one or more reservoirs which are fluidically connected to an inlet of a flow cell or other vessel. The reservoirs can include reagents for use in a method set forth herein. The system can further include a pump, pressure supply or other fluid displacement apparatus for driving reagents from reservoirs to the vessel. The system can include a waste reservoir that is fluidically connected to an egress of a vessel to remove spent reagents. Taking as an example an embodiment where the vessel is a flow cell, reagents can be delivered to the flow cell through a flow cell ingress and then the reagents can flow through the flow cell and out the flow cell egress to a waste reservoir. Accordingly, the flow cell can be in fluidic communication with one or more reservoirs of the system. A fluidic system can include at least one manifold and/or at least one valve for directing reagents from reservoirs to a vessel where detection occurs. Exemplary fluidic apparatus that can be used in a system of the present disclosure include those configured for cyclic delivery of reagents, such as those deployed in nucleic acid sequencing reactions. Exemplary fluidic apparatus are set forth in US Pat. App. Pub. Nos. 2009/0026082 A1; 2009/0127589 A1; 2010/0111768 A1; 2010/0137143 A1; or 2010/0282617 A1; or U.S. Pat. Nos. 7,329,860; 8,951,781 or 9,193,996, each of which is incorporated herein by reference.

The present disclosure provides computer systems (e.g. computer control systems) that are programmed to implement methods, algorithms or functions set forth herein. Optionally, a computer system set forth herein can be a component of a detection system. Optionally, a computer system can be programmed or otherwise configured to: (a) receive an input set forth herein such as signals from a detector, a recognition outcome profile, a database comprising information characterizing or identifying a plurality of candidate glycans or candidate proteins, an interaction model and/or a binding model, (b) determine probabilities for probes interacting with candidate glycans, for example, based on a binding model, (c) identify extant glycans as selected candidate glycans, and/or (c) output an identity or other characteristic of a glycan.

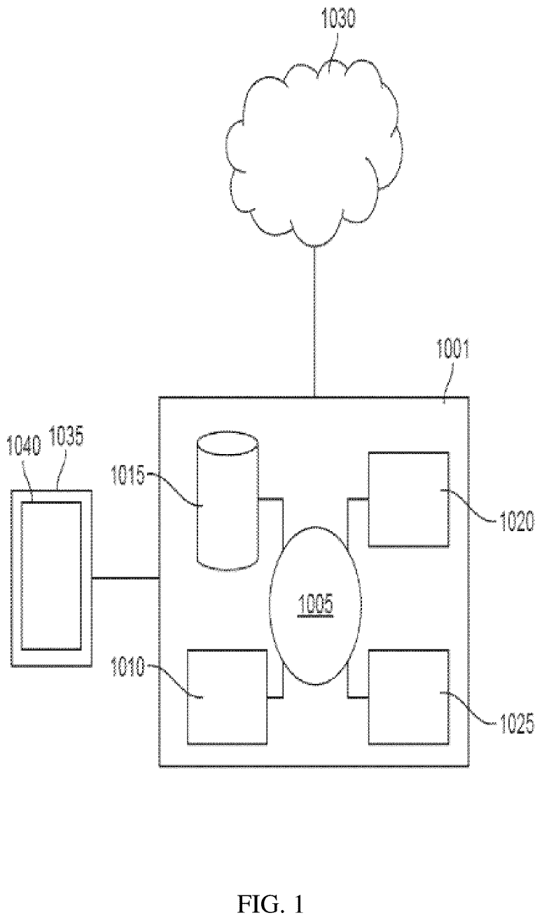

FIG. 1 shows an exemplary computer system 1001. The computer system 1001 can be an electronic device of a detection system, the electronic device being integral to the detection system or remotely located with respect to the detection system. For example, the electronic device can be a mobile electronic device. The computer system 1001 includes a computer processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. For example, one or more computer servers may enable cloud computing over the network 1030 (“the cloud”) to perform various aspects of analysis, calculation, and generation of the present disclosure, such as, for example, receiving information of empirical measurements of extant glycans in a sample; processing information of empirical measurements against a database comprising a plurality of candidate glycans, for example, using a binding model or function set forth herein; generating probabilities of a candidate glycan generating empirical measurements, and/or generating probabilities that extant glycans are correctly identified in the sample. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample.

The present disclosure provides a non-transitory information-recording medium that has, encoded thereon, instructions for the execution of one or more steps of the methods set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

What is claimed is:

1. A method, comprising
(a) providing an array of extant glycans, wherein the array comprises a plurality of addresses, wherein different extant glycans are attached to different addresses of the array;
(b) contacting the array with a plurality of different probes, the different probes recognizing different carbohydrate moieties;
(c) detecting positive recognition outcomes of the plurality of different probes at individual addresses of the array, thereby producing outcome profiles for the addresses;
(d) providing a database comprising a set of candidate glycans, the database comprising, for each candidate glycan, the probability of a positive recognition outcome for the plurality of different probes; and
(e) determining with a computer, using the database and the outcome profiles, candidate glycans in the database corresponding to different extant glycans in the array.

2. The method of claim 1, wherein step (c) further comprises detecting negative recognition outcomes of the plurality of different probes at individual addresses of the array, whereby the outcome profiles for the addresses comprise the positive recognition outcomes and the negative recognition outcomes.

3. The method of claim 2, wherein the database further comprises, for each candidate glycan, the probability of a negative recognition outcome for the plurality of different probes.

4. The method of claim 2, wherein the positive recognition outcomes comprise positive binding outcomes, and wherein the negative recognition outcomes comprise negative binding outcomes.

5. The method of claim 1, wherein the different extant glycans are attached to glycoproteins, glycolipids, or cells at the different addresses of the array.

6. The method of claim 1, wherein the plurality of different probes comprises carbohydrate binding reagents, and wherein the positive recognition outcomes comprise positive binding outcomes.

7. The method of claim 1, further comprising contacting the array with a glycan modifying reagent.

8. The method of claim 7, wherein the glycan modifying reagent comprises an N-glycosidase or endoglycosidase, hydrazine, ammonia, ammonium carbonate, N-bromosuccinimide, sodium hypochlorite, sodium hydroxide, or sodium borohydride.

9. The method of claim 7, further comprising, after contacting the array with the glycan modifying reagent:
(f) contacting the array with a second plurality of different probes, the different probes recognizing different carbohydrate moieties; and
(g) detecting positive recognition outcomes of the second plurality of different probes at individual addresses of the array, thereby producing second outcome profiles for the addresses.

10. The method of claim 1, wherein step (e) comprises determining with the computer, using the database and the outcome profiles, the most probable candidate glycan or most probable group of candidate glycans corresponding to different extant glycans in the array.

11. The method of claim 1, wherein the computer uses a machine learning algorithm to determine the candidate glycans in the database corresponding to different extant glycans in the array.

12. The method of claim 1, wherein the determining of step (e) comprises calculating, for an individual address of the array, a probability that the outcome profile is observed given that a candidate glycan in the database is present at the individual address.

13. The method of claim 12, wherein the calculating, for the individual address of the array, is performed for a plurality of candidate glycans in the database.

14. The method of claim 13, wherein the calculating is performed for a plurality of individual addresses in the array.

15. The method of claim 1, wherein step (e) comprises determining a probability or likelihood that one or more candidate glycan in the database would generate one or more of the recognition outcomes for the extant glycan attached to one or more of the addresses of the array.

16. The method of claim 1, wherein individual probes in the series of probes are each promiscuous for a subset of different glycans in the array.

17. The method of claim 16, wherein the individual probes each comprise a carbohydrate binding reagent that recognizes two or more different carbohydrate epitopes present in the different extant glycans in the array.

18. A method, comprising
(a) providing an array of extant glycoconjugates, wherein the array comprises a plurality of addresses, wherein different extant glycoconjugates are attached to different addresses of the array;
(b) contacting the array with a plurality of different probes, the different probes recognizing different carbohydrate moieties;
(c) detecting positive recognition outcomes for the plurality of different probes at individual addresses of the array, thereby producing outcome profiles for the addresses;
(d) providing a database comprising a set of candidate glycans or glycoconjugates, the database comprising, for each candidate glycan or glycoconjugate, the probability of a positive recognition outcome for the plurality of different probes; and
(e) determining with a computer, using the database and the outcome profiles, candidate glycans or glycoconjugates in the database corresponding to glycans or glycoconjugates at addresses of the array.

19. A system for detecting glycans, comprising
(a) a detector configured to acquire signals from a plurality of interactions occurring between a plurality of different probes and a plurality of different extant glycans in a sample;
(b) a database comprising information characterizing or identifying a plurality of candidate glycans;
(c) a computer processor configured to:
(i) communicate with the database,
(ii) process the signals to produce a plurality of outcome profiles, wherein each of the outcome profiles comprises a plurality of recognition outcomes for interaction of an extant glycan of (a) to the plurality of different probes, wherein individual recognition outcomes of the plurality of recognition outcomes comprise a measure of interaction between an extant glycan of (a) and a different probe of the plurality of different probes,
(iii) process the recognition profiles to determine a probability for each of the probes interacting with each of the candidate glycans in the database according to an interaction model for each of the probes; and
(iv) output an identification of selected candidate glycans, the selected candidate glycans being candidate glycans in the database having a probability for interaction with each of the probes that is most compatible with the plurality of recognition outcomes for the extant glycans.

20. A system for detecting glycoproteins, comprising
(a) a detector configured to acquire signals from a plurality of reactions occurring between a plurality of different probes and a plurality of different extant glycoproteins in a sample;
(b) a database comprising information characterizing or identifying a plurality of candidate glycans or glycoproteins;
(c) a computer processor configured to:
(i) communicate with the database,
(ii) process the signals to produce a plurality of outcome profiles, wherein each of the outcome profiles comprises a plurality of recognition outcomes for interaction of an extant glycoprotein of (a) to the plurality of different probes, wherein individual recognition outcomes of the plurality of recognition outcomes comprise a measure of interaction between an extant glycoprotein of (a) and a different probe of the plurality of different probes,
(iii) process the outcome profiles to determine a probability for each of the probes interacting with each of the candidate glycans in the database according to a trained model for each of the probes; and
(iv) output an identification of selected candidate glycans or glycoproteins, the selected candidate glycans or glycoproteins being candidate glycans or glycoproteins in the database having a probability for interaction with each of the probes that is most compatible with the plurality of recognition outcomes for the extant glycoproteins.
